(19) United States
(12) Patent Application Publication
(10) Pub. No.: US 2024/0342075 A1
(43) Pub. Date: Oct. 17, 2024

(54) DRUG DELIVERY PLATFORM FOR DELIVERY OF THERAPEUTICS AND METHODS OF USE AND MANUFACTURE THEREOF

(71) Applicant: Suzanne Ahmed, Jamestown, NC (US)

(72) Inventor: Suzanne Ahmed, Jamestown, NC (US)

(21) Appl. No.: 18/292,036

(22) PCT Filed: Jul. 25, 2022
(86) PCT No.: PCT/US22/38226

§ 371 Date: Jan. 25, 2024

Related U.S. Application Data
(60) Provisional application No. 63/226,753, filed on Jul. 28, 2021.
(60) Provisional application No. 63/225,923, filed on Jul. 26, 2021.

(51) Int. Cl. A61K 9/46; A61K 9/16; A61K 31/37; A61K 31/422; A61K 31/5415; A61K 38/28; A61K 47/02; A61K 47/12; A61K 47/38

(52) U.S. Cl. CPC A61K 9/0007; A61K 9/1652; A61K 9/1676; A61K 31/37; A61K 31/422; A61K 31/5415; A61K 38/28; A61K 47/02; A61K 47/12; A61K 47/38

(57) ABSTRACT

Disclosed is a holistic platform for the delivery of drugs across tissues and biological barriers such as the blood brain barrier, a gastrointestinal barrier or the like for enhanced drug absorption. In this invention, pharmaceutical compositions, methods of administration, and manufacture of a pharmaceutical composition configured to physically penetrate and/or embed an active ingredient (e.g., a pharmaceutical agent) within tissues or a biological barrier is described. The pharmaceutical composition is configured to increase the physical crossing of the pharmaceutical agent across the biological barrier promoted by the production of a propulsion gas to drive the pharmaceutical agent into the barrier. The pharmaceutical compositions and methods can be used to deliver a pharmaceutical agent across a plurality of biological barriers, thereby increasing the penetration, retention time, and absorption of the pharmaceutical agent.

### CROSS-REFERENCE TO RELATED APPLICATIONS

This application claims priority from U.S. Provisional Application No. 63/225,923, filed on 26 Jul. 2021, which is incorporated by reference herein in their entirety for all purposes.

### BACKGROUND

Drug delivery across biological barriers can present a formidable challenge to the treatment of various diseases. The ability of drugs to penetrate and permeate these barriers can determine the clinical outcome of various therapeutic treatments. To circumvent some of these challenges, low permeability drugs are often administered parenterally; yet even parentally, barriers such as the blood brain barrier are not circumvented. Parenteral administration presents several challenges, primary among them is the relatively low patient compliance due to fear of needles and avoidance of pain. Additionally, in some cases, parenteral administration needs to be done in a clinical setting or with the help of someone other than the patient when self-administration is not feasible, hence reducing patient independence. Parenteral administration also carries a risk of infection at the site of injection.

Nasal drug delivery offers a non-invasive drug delivery route amenable to self-administration that also by-passes many of the metabolic processes encountered by a therapeutic administered via other routes such as the oral route. Additionally, nasal delivery offers the potential of more patient compliance than a parenteral route as it is pain free. Nasal drug administration enables delivery to patients who may have difficulty swallowing including geriatric and pediatric as well as patients who may be unconscious. Most importantly, nasal delivery also offers a method for the direct delivery of therapeutics to the brain (nose-to-brain delivery) or the central nervous system potentially across the blood brain barrier (BBB).

Drug delivery across the blood brain barrier presents a formidable challenge for the treatment of CNS diseases. While multiple methods are in development to assist therapeutic agents in crossing this barrier on average more than 90% of molecules developed for the treatment of various diseases (including cancers) are unable to be used for the treatment of brain and CNS disorders due to the difficulty of delivery across the blood brain barrier.

Oral (or sublingual) drug delivery represents the highest compliance route of drug administration. Yet it remains a formidable challenge to deliver low permeability therapeutics, including peptides, proteins and biologics, orally due to the gastrointestinal barrier.

In all cases, improving the absorption and bioavailability of drugs, even for drugs that are currently delivered via a nasal or oral route, would potentially assist in the reduction of the dose needed and hence a reduction in the occurrence or severity of side effects.

In this invention a novel, therapeutic-agnostic holistic platform technology is presented that improves the penetration of drugs into a biological barrier or tissue, hence improving retention, absorption and bioavailability. Pharmaceutical agents will be physically propelled to embed within or penetrate a biological tissue or biological barrier. This will be accomplished by physically propelling and driving the active pharmaceutical ingredient containing particles into and across a barrier via the asymmetric release of a biologically benign gas from a coating of the particles resulting in a transfer of momentum to the particles propelling and moving them into the barrier as will be described in detail below.

Particles with propulsive capability have primarily been studied in the academic realm. They tend to rely on materials and propulsion mechanisms that are not conducive to use in biological system. Many of the particles have metallic elements. Motion of many of the particles relies on the breakdown of a chemical fuel that is not biocompatible. Other propulsion mechanisms rely on external magnetic or electromagnetic fields rendering their use not feasible for applications in the case of self-administration by a patient because a clinical setting with specialized equipment would be required. Generally, there is as a lack of an overall method or paradigm of how particles with propulsive capability will be incorporated into drug administration.

The fabrication of these particles with propulsive capability typically relies on highly specialized equipment with no path to regulatory or GMP approval. This equipment is expensive, highly specialized and the synthesis of these particles tends to be non-scalable, expensive and not suited for drug product manufacturer. Hence there is a lack of commercial manufacturing approaches for propulsive particles to enable their realistic and cost-effective use in applications.

In this invention we remedy this situation by introducing a full commercial manufacturing approach using regulatory agency-approved pharmaceutical unit operations for the large-scale fabrication of biocompatible particles with propulsive capability that rely on a self-contained biocompatible propulsion mechanism that allows patient self-administration.

In this invention enhanced drug delivery and in particular, delivery across a biological tissue or a biological barrier, such as the blood brain barrier or gastrointestinal barrier, will be accomplished by physically propelling and driving an entity containing an active pharmaceutical agent into and across a biological tissue or a biological barrier via the asymmetric release of a biologically benign gas from a coating of the particle, resulting in a transfer of momentum to the pharmaceutical. The transfer of momentum propels and moves the pharmaceutical into the barrier, described herein. Additionally, this invention provides methods of administration and introduces a full commercial manufacturing approach using regulatory agency-approved pharmaceutical unit operations for the large-scale fabrication of biocompatible particles with propulsive capabilities that rely on a self-contained biocompatible propulsion mechanism to allow patient self-administration.

### SUMMARY

This disclosure relates to the field of drug delivery with the introduction of a novel approach for drug delivery of therapeutics. It also relates to drug delivery across a biological barrier, e.g., blood brain barrier, gastrointestinal barrier, and the like. Described herein is a drug delivery platform for the powered, active delivery of drugs across biological tissue or a biological barrier. Disclosed are compositions for propelling drugs, or compositions, such as particles, containing the drugs, across one or more biological barriers in order to improve systemic or local delivery thereof, and methods of their manufacture.

This invention presents a therapeutic-agnostic platform technology for the delivery of therapeutics for increased absorption and bioavaliability with an emphasis on drug delivery across biological barriers and tissues. This invention describes the composition, method of administration and manufacture of a delivery platform that increases the physical penetration, retention and absorption of the drug through biological tissues via a propulsive mechanism.

The use of a penetrating physical transport method to cross biological barriers such as the BBB offers multiple advantages over chemical and biological modifications of the active drug to improve absorption.

In some embodiments of the invention, the entity containing a pharmaceutical therapeutic, such as a neat active pharmaceutical ingredient powder, as an active pharmaceutical ingredient-containing particle or as granules of either; is admixed with or coated with a propulsion agent comprising a component capable of evolving a propulsion gas [e.g., a carbonate or bicarbonate salt] upon contact with an activation agent [e.g. water, acidic solution, saliva, gastric fluid or the like]; wherein allowing the pharmaceutical agent to embed within or penetrate a biological tissue or biological barrier.

In some embodiments of the invention, the propulsion gas evolved is a biologically benign gas or a gas, that at the evolved amount, does not pose a hazard to the health of the patient, be it a human or an animal; wherein the propulsion gas can be one or more of CO₂, N₂, O₂, NO₂, H₂, H₂O vapor and SO₂.

In some embodiments of the invention, the component of the propulsion agent capable of evolving a propulsion gas can be a water-insoluble or water-soluble compound. In some embodiments of the invention, the component of the propulsion agent capable of evolving a propulsion gas comprises a carbonate or bicarbonate compound, a nitrate or nitrite compound, a sulfite compound or the like.

In some embodiments of the invention, the activation agent may be water. In some embodiments of the invention, the activation agent may be an acidic solution of a pH below about 7. An acidic solution activation agent solubilizes or loosens one or more biological structures, e.g., mucus, to allow for further penetration of the pharmaceutical across the barrier. In some embodiments of the invention, the activation agent may be a solution of pH in the range of about 1 to about 14. In some embodiments of the invention, the activation agent may be a fluid already existing within the body [e.g. gastric fluids, saliva and the like].

In some embodiments of the invention, the active pharmaceutical ingredient (API) may exist as neat API particles or exist within API containing particles that can contain pharmaceutically acceptable excipient such as polymers, lipids, carbohydrates, or a combination thereof. These particles can be prepared using manufacturing techniques such as spray drying, lyophilization, freeze-lyophilization, freeze spray drying, solvent evaporation or any other regulatory approved manufacturing technique as is known by those skilled in the art. These particles can me manufactured with excipients co-lyophilized, co-spray-dried or co-processed with the active ingredient, forming a matrix in which the drug is dispersed, or forming a protective shell around the active ingredient such as in microcapsules and liposomes

Exemplary routes of administration of the pharmaceutical compositions include nasal, oral (e.g. gastrointestinal), sublingual, rectal, ocular administration, transdermal administration, and the like. In some embodiments, a pharmaceutical is provided via oral administration. In some embodiments, a pharmaceutical is provided via nasal administration. In some embodiments, a pharmaceutical is provided by mucosal administration, rectal administration, ocular administration, vaginal administration, transdermal administration, and the like.

In some embodiments of the invention, the pharmaceutical composition, formulated with active pharmaceutical ingredients and excipients, admixed with or coated with a gas releasing propulsion agent (e.g. carbonate), is administered nasally, wherein the evolution of the propulsion gas is triggered by the administration of an activation agent (e.g. an acidic solution spray or water spray). Hence the pharmaceutical composition is first administered in the nasal cavity. After this first administration, the activation agent is sprayed into the same region of the nasal cavity and comes into contact with the previously administered pharmaceutical composition, promoting the asymmetric release of gas from the exposed region of the pharmaceutical composition adhered to the nasal lining, which promotes embedding or penetration of all or a part of the pharmaceutical composition into the biological tissue or a biological barrier.

In some embodiments of the invention the nasal dosage forms of the pharmaceutical composition can be in the form of powders, sprays, emulsions, suspensions, and the like, which contain the active ingredient(s) in a mixture with non-toxic pharmaceutically acceptable excipients.

In some embodiments of the invention, the time duration between the administration of the pharmaceutical composition and the activation agent spray for nasal administration can be fractions of a second, a second or seconds, minutes, an hour or more than an hour

In some embodiments of the invention, the pharmaceutical composition with a gas releasing propulsion agent and the activation agent for nasal administration can be housed in the same or separate devices.

In some embodiments of the invention, administration of the pharmaceutical composition nasally may be used for nose-to-brain delivery of the pharmaceutical agent(s), the systemic delivery of the pharmaceutical agent(s) or delivery of the pharmaceutical agent(s) to non-brain target organs.

In some embodiments of the invention, the pharmaceutical composition is an internasal powder comprising, one or more active pharmaceutical ingredients, one or more gas producing propulsion agent compounds, One or more acids in powder form to allow the use of a neutral aqueous spray as an activation agent or to increase the rate of gas production, One or more mucoadhesive agents (e.g. hydroxypropyl cellulose, hydroxypropyl methyl cellulose, chitosan, and the like), One or more diluents (e.g. microcrystalline cellulose, lactose, and the like), One or more disintegrants or super disintegrants (e.g. croscarmellose sodium, crospovidone and the like), Absorption enhancers and other pharmaceutically acceptable excipients.

In some embodiments of the invention, the pharmaceutical composition, formulated with active pharmaceutical ingredients and excipients, admixed with or coated with a gas releasing propulsion agent (e.g. carbonate), is administered orally, wherein the activation agent(s) are gastric fluids already present within the gastrointestinal tract. Evolution of the propulsion gas is triggered by contact with these fluids (e.g. an acidic solution spray or water spray) which promotes embedding or penetration of all or a part of the pharmaceutical composition in a biological tissue or a biological barrier.

In some embodiments of the invention, the pharmaceutical compositions for oral administration (oral dosage forms) may comprise tablets, capsules, a liquid solution or suspension, a powder, or liquid or solid crystals, which contain an active ingredient(s), propulsion agent(s) comprising a component capable of evolving a propulsion gas a propulsion, in a mixture with non-toxic pharmaceutically acceptable excipients.

In some embodiments of the invention, the pharmaceutical composition is an oral tablet of a powder comprising: one or more active pharmaceutical ingredients, one or more diluents (e.g. microcrystalline cellulose, lactose, mannitol, starch and the like), one or more disintegrants or super disintegrants (e.g. sodium starch glycolate, croscarmellose sodium, crospovidone and the like) to ensure the breakup of a tablet formulation, one or more lubricants, anti-adherents and glidants (e.g. magnesium stearate, stearic acid, and the like), one or more gas producing propulsion agents (e.g. carbonate, bicarbonate and the like), potentially one or more acids in powder form to lower the local pH surrounding the tablet increasing the rate of gas production in the case of both soluble or insoluble gas producing propulsion agents, potentially one or more degradation enzyme inhibitors and potentially one or more permeation enhancers.

In some embodiments of the invention, an oral tablet may be enterically coated or the powder may be loaded into a capsule or an enteric capsule.

In some embodiments of the invention, the pharmaceutical composition, formulated with active pharmaceutical ingredients, excipients such as diluents and a gas releasing propulsion agent, is administered sublingually, wherein the activation agent(s) are moisture already present under the tongue. Evolution of the propulsion gas is triggered by contact with this moisture (e.g. saliva) which promotes embedding or penetration of all or a part of the pharmaceutical composition in a biological tissue or a biological barrier.

In some embodiments of the invention, the sublingual dosage forms of the pharmaceutical composition can be in the form of powders, sprays, tablets, films and the like, which contain the active ingredient(s) in a mixture with non-toxic pharmaceutically acceptable excipients.

While this invention represents a therapeutic-agnotistic platform, illustrative examples of drugs that can utilize the present invention for nasal delivery include sumatriptan succinate, zolmitriptan salts, naratriptan, riza-triptan, almotriptan, eletriptan, frovatriptan, bupivacaine, fibroblast growth factor, cephalexin, lidocaine, clobazame, midazolam, alprazolam, diazepine, lorazepam, dexmedeto-65 midine, monosialoganglioside, cocaine, insulin, glucagon, oxytocin, fentanyl, sulfentanil, diamorphine, ketamine, apo-morphine, buprenorphine, morphine sulphate, oxycodone hydrochloride, butorphanol, NSAIDs, paracetamol, benzo-diazepines, dopamine, pramipexole, rasagiline, rogitine, ondansetron, granisetron, metoclopramide, naloxone, naltrexone, atropine, adrenaline, cannabis active compounds, epinephrine, isosorbide dinitrate, obitoxine, dexmedetomi-dine, metochlorpramide, L-dopa, nicotine, sildenafil, nafarelin, dobutarnine, phenylephrine, tramazoline, xylometazo-line, tramadol, methacholine, ipratropium, scopolamine, propranolol, verapamil, hydralazine, nitroglycerin, clofilium tosylatecannabis active compounds and pharmaceutically acceptable salts, isomers, and mixtures thereof.

These drugs span a wide range of indications including use in common cold treatment, anti-addiction agents, anti-infective agents, analgesics, anaesthetics, antarthritics, anti-allergy agents, antiasthmatic agents, anticonvulsants, anti-depressants, antidiabetic agents, anti-diuretics, anti-emetics, antihistamines, anti-hypertensive agents, anti-inflammatory agents, antimigraine preparations, anti-motion sickness preparations, antinauseants, antineoplastics, anti-obesity, antiosteoporosis, anti-Parkinsonism drugs, antipru-ritics, antipsychotics, antipyretics, anticholinergics, benzodiazepine antagonists, bone stimulating agents, central nervous system stimulants, hormones, hypnotics, immuno-suppressives, prostaglandins, proteins, peptides, polypep-tides and other macromolecules, psychostimulants, com-pounds for use in rhinitis treatment, compounds for use in sexual hypofunction treatment, sedatives, compounds for use in treatment of known or suspected opioid overdose, tranquilizers and vitamins, probiotics, natural ingredients, peptide or protein therapeutic agents such as cytokines, hormones, clotting factors, vaccines, monoclonal antibodies, amino acids, or any combination thereof.

In some embodiments of the invention, and in particular those that are nasally administered, have the potential to have a significant impact on nose-to-brain delivery and potentially delivery across the blood brain barrier. Hence will have a significant impact on the treatment of brain, CNS and neurological disorders, or complications resulting therefrom. Non-limiting examples of neurological disorders include multiple sclerosis (MS), ischemia, amyotrophic lateral sclerosis (ALS), Parkinson's disease (PD), Alzheimer's disease (AD), autism, Rett syndrome, Fragile X syndrome, Angelman syndrome, cerebral palsy, Down syndrome, Dravet syndrome, epilepsy schizophrenia, bipolar disorder, anxiety disorder, major depressive disorder (MDD), and the like.

Additional illustrative examples of drugs that are suitable for oral administration or sublingual administration utilizing the present invention include antibacterial/anti-infective agents, such as ciprofloxacin, cefuroxime, cefatrizine, cefpodoxime, clarithromycin, loracarbef, azithromycin, cefixime, cefadroxil, amoxycillin, and the like; antivirals, such as acyclovir; cardiovascular agents, such as diltiazem, captopril and the like; non-steroidal anti-inflammatory agents, such as etodolac, ketorolac, and the like; anti-ulcer agents, such as ranitidine, famotidine, and the like; drugs for respiratory diseases, such as fexofenadine, pseudoephe-drine, phenylpropanolamine, dextromethorphan, chlorphe-30 niramine, and the like; dopaminergic agents, such as bro-mocriptine; immunosuppressants, such as cyclosporin; skeletal muscle relaxants, such as baclofen; anti-gout agents, such as allopurinol; and the like.

While this invention represents a therapeutic-agnostic platform, illustrative examples of drugs that can utilize the present invention for drug delivery include therapeutics which can be classified as small molecules, macromolecules (e.g. a peptide, protein or biologic), BCS I class drugs, BCS II class drug, BCS III class drug and BCS VI class drugs.

In some embodiments of the invention, a manufacturing process for the production of a powder of composition with an active pharmaceutical ingredient(s) coated with the gas releasing propulsion agent via simple mechanofusion includes:
i) sieving each individual component of the formulation to ensure the removal of any clumping
ii) Blending of the components which includes the active pharmaceutical ingredient(s) and the gas producing propulsion agent, and can include diluents, disintegrants and lubricants if needed.
   wherein blending of the active pharmaceutical ingredient(s) and the gas producing propulsion agent leads to the mechanofusing of the different particles, where they adhere due to surface forces which are especially magnified for particles on the micron, sub-millimeter and millimeter scale.

In some embodiments of the invention, a manufacturing process for the production of a powder composition incorporating dry granulation or agglomeration for nasal, oral or sublingual administration, where active pharmaceutical ingredient(s) containing granules both contain and are coated with the gas releasing propulsion agent comprises:
i) Sieving each individual component to ensure the removal of any clumping
ii) Blending of the pre-granulation components which includes the active pharmaceutical ingredient(s) and gas producing propulsion agent, and can include diluents, and other excipients such as disintegrants and lubricants as needed.
iii) Dry Granulation or Agglomeration (e.g. roller compaction, mechanical agglomeration using a sieve shaker or the like)
iv) Coating granules with gas producing propulsion agent. This may be done using simple blending via a mechanofusing mechanism or via other methods such as film coating. Optional additional excipients may be added here.
v) Sieving to remove any excess gas producing propulsion agent (with this being an optional step)
vi) Blending with any other excipients (optional step and may be done to assist in compaction into tablets for oral or sublingual administration)
   The resulting powder can be: i) loaded into a delivery device for nasal administration, ii) loaded into a capsule that is either enteric or non-enteric for oral administration, iii) compacted into tablets that can either be coated with an enteric or non-enteric coating, or not coated for oral administration or iv) compacted into tablets for sublingual administration.

In some embodiments of the invention, a manufacturing process to produce active pharmaceutical ingredient-containing granule that is just coated with the gas releasing propulsion agent process comprises:
i) Sieving each individual component to ensure the removal of any clumping
ii) Blending of the pre-granulation components which includes the active pharmaceutical ingredient(s) and can include diluents, disintegrants and lubricants if needed.
iii) Dry Granulation or Agglomeration (e.g. roller compaction, mechanical agglomeration using a sieve shaker or the like)
iv) Coating granules with gas producing propulsion agent. This may be done using simple blending via a mechanofusing mechanism or via other methods such as film coating. Optional additional excipients may be added here.
v) Sieving to remove any excess gas producing propulsion agent (with this being an optional step)
vi) Blending with any other excipients (optional step and may be done to assist in compaction into tablets for oral or sublingual administration)

In some embodiments of the invention, a manufacturing process as described above where wet granulation is used rather than dry granulation.

In some embodiments of the invention, a process for the production of oral or sublingual tablets of the pharmaceutical compositions by utilizing tablet compaction.

In some embodiments of the invention, a process for the coating of tablets with enteric or non-enteric capsules utilizing a film coating process.

In some embodiments, for propulsion agents that are insoluble (e.g. some carbonates), they may be administered as an intranasal spray where the particles are in a PH neutral suspension, so long as the adherence of the salt to the particles is not affected by their presence as a suspension. Intranasal powder tends to offer improved chemical stability for the API.

### Definitions

To facilitate the understanding of the present disclosure, a number of terms are defined below. Terms defined herein have meanings as commonly understood by a person of ordinary skill in the areas relevant to the disclosure. Terms such as “a,” “an,” and “the” are not intended to refer to only a singular entity, but include the general class of which a specific example may be used for illustration. The terminology herein is used to describe specific embodiments of the disclosure, but their usage does not limit the disclosure, except as outlined in the claims.

The terminology used herein is for describing particular embodiments only and is not intended to be limiting of the invention. The term “comprising” and “comprises”, used in the claims, should not be interpreted as being restricted to the components and steps listed thereafter; they do not exclude other components or steps. They need to be interpreted as specifying the presence of the stated features, integers, steps and/or components as referred to, but does not preclude the presence and/or addition of one or more other features, integers, steps or components, or groups thereof. Thus, the scope of the expression “a composition comprising A and B” should not be limited to compositions consisting only of components A and B. Also, the scope of the expression “a method comprising the steps X and Z” should not be limited to methods consisting exclusively of those steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum.

As used herein, the terms “administer” and “administering” are used to indicate the process of providing a therapeutic, pharmaceutical, housing compartment, medication, or the like thereof to a subject. In some embodiments, a pharmaceutical is provided via oral administration. In some embodiments, a pharmaceutical is provided via nasal administration. In some embodiments, a pharmaceutical is provided by mucosal administration, rectal administration, ocular administration, vaginal administration, transdermal administration, and the like.

As used herein, the term “pharmaceutical composition” refers to an active compound, formulated together with one or more pharmaceutically acceptable excipients. In some embodiments, a compound of the disclosure is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid form, including those adapted for oral administration, for example, tablets, pills, capsules, or other form targeted for oral, buccal, sublingual, and systemic absorption, e.g., boluses, powders, particles, or granules, for application to the tongue. In some embodiments, pharmaceutical compositions may be specially formulated for administration in solid form suspended in one or more liquids, including those adapted for nasal administration, for example, emulsions or suspensions containing a solid form.

The terms “active agent”, “pharmaceutical active agent”, “active”, “API”, “active pharmaceutical ingredient”, “active substance”, “active molecule”, “active compound”, “pharmaceutical agent” or “drug” are used interchangeably.

An “API entity” as used herein, refers to any entity that contains one or more active pharmaceutical ingredient(s) which can include a neat active pharmaceutical ingredient particle, a particle in which the active pharmaceutical ingredient is a component such as with particles produced as a result of co-spray drying, co-lyophilization or any other form of co-processing with an excipient(s), a particle in which the active pharmaceutical ingredient is encapsulated such as a liposome, or a granule or an aggregate of the active pharmaceutical ingredient(s) with or without excipients such as those that can be produced using granulation approaches such as roller compaction.

The terms “soluble” and “insoluble” refer to a compound's solubility in water.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: anti-adherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art.

A “propulsion agent,” as used herein, refers to a gas producing compound, e.g. salt, that is a biocompatible agent and approved by regulatory agencies such as the FDA or the EMA for use in pharmaceutical compositions. The gas producing compound may include any compound that produces a gas that is non-hazardous at the produced amounts or biocompatible with an organism and/or chemically safe to use as denoted by regulatory agencies such as the FDA or the EMA or the like. In some embodiments, the gas-producing compound may be a carbon dioxide producing compound, e.g., carbonates and bicarbonates; a nitrogen dioxide or nitrogen producing compound, e.g. nitrates or nitrites, a hydrogen producing compound, an oxygen producing compound or the like. Carbonates may include any form of soluble or insoluble carbonates, such as magnesium carbonate, calcium carbonate, iron (II) carbonate, sodium carbonate, potassium carbonate, of the like thereof. Examples of bicarbonates that may be used as propulsion agents include sodium bicarbonate, potassium bicarbonate, magnesium bicarbonate, calcium bicarbonate, ammonium bicarbonate, carbonic acid, or the like. The gas-producing compound would release a chemical gas when coming into contact with the activation agent to propel and embed one or more particles into a biological barrier.

As used here, the term “activation agent” refers to one or more compounds capable of triggering the release of a gas upon contact with a propulsion agent such that a chemical in a gas form is emitted. For example, an activation agent may include an aqueous solution that solubilizes a propulsion coating of a carbonate such that the propulsion agent may react with its own compounds and emit a chemical in a gas form, e.g., CO₂. The activation agent may be one or more biological fluids, e.g., gastric juices, salivary fluids, interstitial fluids, extracellular fluids, intracellular fluids, or the like. The activation agent may be an acidic solution that is capable of solubilizing both soluble and insoluble forms of propulsion agents such as those that are comprised of compounds having a carbonate structure.

In some embodiments, the activation agent may be water. In some embodiments of the invention, the activation agent may be an acidic solution of a pH below about 7. An acidic solution activation agent solubilizes or loosens one or more biological structures, e.g., mucus, to allow for further penetration of the pharmaceutical across the barrier. In some embodiments, the activation agent may be a solution of pH in the range of about 1 to about 14. In some embodiments, the activation agent may react with the propulsion agent such that a unidirectional release of a chemical in the form of a gas will occur from the exposed region of the particles and propulsion into the barrier will occur.

The term “propulsion gas,” as used herein, refers to a biocompatible chemical that exists in a gaseous form at temperatures ranging from about 0° C. to 100° C. The propulsion gas may include any form of biocompatible chemical, or non-toxic chemical at the released amount, that may be produced as a function of a chemical reaction or physical interaction between an activation agent and a propulsion coating. The propulsion gas may be, e.g., carbon dioxide, nitrogen, hydrogen, oxygen, water vapor, nitrogen dioxide and sulfur dioxide in small amounts depending on the location of the biological barrier within the body.

### BRIEF DESCRIPTION OF FIGURES

FIGS. 1A-1D are schematic representations of compositions containing a pharmaceutical agent(s) that are coated with a propulsion coating. FIG. 1A. shows a propulsion coating 100 and a pharmaceutical agent 101, e.g., which can be in the form of a granule, aggregate, particle, or powder 101. FIG. 1B shows a propulsion coating that is present within a pharmaceutical granule 102 and containing the pharmaceutical agent 101. FIG. 3C shows a composition containing a pharmaceutical agent that is adhered to a biological barrier or biological tissue. 102. The arrow represents the addition of an activation agent with the release of a gas (e.g., CO₂) as indicated with the “-” sign. FIG. 1D shows a schematic of the gas released 103 and subsequent propulsion of the entity that contains a pharmaceutical agent into the biological barrier or biological tissue. 102. (wherein the arrow indicates direction of motion caused by the propulsion).

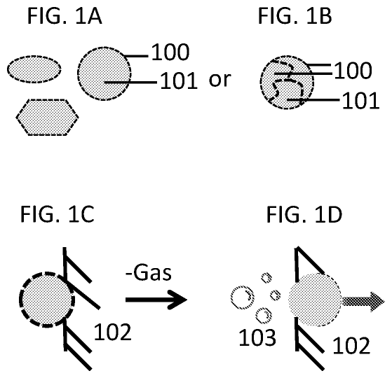

FIGS. 2A and 2B are schematics showing intranasal administration of a composition containing at minimum a pharmaceutical agent and a gas releasing propulsion agent (FIG. 2A) followed by administration of an activation agent (FIG. 2B) that interacts with the previously administered gas releasing propulsion agent of the composition, resulting in the evolution of gas for propulsion and embedding or penetration of the pharmaceutical agent into the nasal tissue lining.

FIGS. 3A-3E are schematic representations of compositions containing a pharmaceutical agent(s) that are coated with a gas releasing propulsion agent coating and method of administration. FIG. 3A. shows a propulsion agent coating 100 and a composition containing a pharmaceutical agent(s) 101, e.g. which can be in the form of a granule, aggregate, particle, or neat pharmaceutical agent particle 101. FIG. 3B shows a propulsion agent that is present within a pharmaceutical granule 100 and containing the pharmaceutical agent 101. FIG. 3C shows a tablet dosage form containing a pharmaceutical composition that is coated with a gas releasing propulsion agent for oral or sublingual self-administration by a subject; in which the blue arrow represents the tablet being taken in by the subject. FIG. 3D shows tablets containing a pharmaceutical agent(s) that is released within the GI tract or mouth 300 upon the disintegration of the host oral dosage form (e.g., a tablet or capsule), in which motion of the composition or components thereof is generated by a propulsion gas 301, e.g., carbon dioxide, that is released when the propulsion agent within the dosage form comes into contact with an activation agent (e.g. gastric fluids or moisture in the mouth). FIG. 3E. shows propulsion of a composition or components thereof containing a pharmaceutical agent(s) that becomes embedded within the lining of the GI tract or the sublingual lining due to the release of the propulsion gas propulsion agent coating 100, in which the propulsion agent coating 100 is reduced in thickness as gas is evolved.

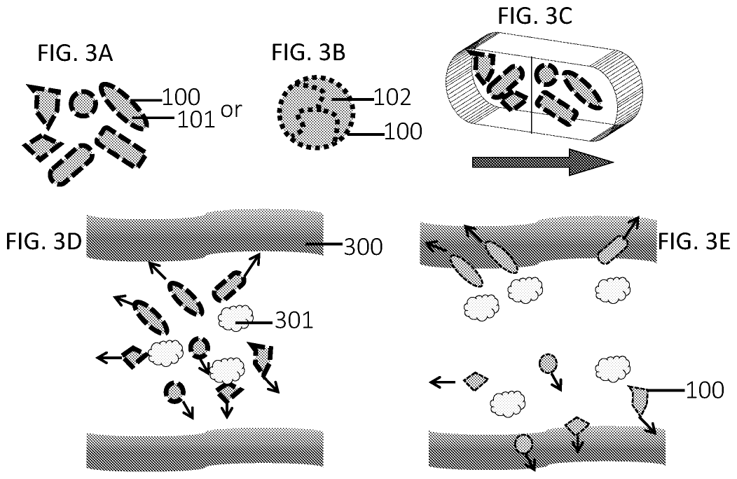

FIGS. 4A-4D are stereomicroscope images of powders used. FIG. 4A. shows microcrystalline cellulose powder. FIG. 4B shows magnesium carbonate (MgCO3) powder which is the propulsion agent. FIG. 4C shows Erioglaucine the model small molecule used for transport experiments. FIG. 4D shows the full composition with all three powders mixed at defined proportions. It shows the mechanofusing of powders. 401 is the model small molecule Erioglaucine the transport of which across a barrier was measured. 402 is the propulsion agent coating the surface of the Eriglaucine. 403 is microcrystalline cellulose adhered to the other powders.

FIGS. 5A-5B show the experimental set up to measure transport across a membrane and the results of the experiment. FIG. 5A shows the vertical Franz cell apparatus used. 501 shows the location of the membrane. 502 shows the donor chamber where the composition with the compound of interest is placed. 503 is the receptor chamber into which the composition permeates. 504 is the sampling port. 505 is the stir bar. FIG. 5B shows the data for the permeation of the Erioglaucine containing composition across a model of a biological barrier (polysulfone). The plot shows the percentage Erioglaucine to cross the barrier as a function of time when an activation agent is applied to the propulsion agent containing composition (triangles and solid trendline) with propulsion, and when no activation agent is applied to the same composition (squares and dashed trendline) without propulsion. With propulsion the composition is detected in the receptor chamber immediately within the first second, while without propulsion transport across the membrane only starts to occur after 4 minutes. The overall transport rate across the membrane as indicated by the slopes is more than 2 times faster with propulsion than without.

### DETAILED EMBODIMENTS

Disclosed is a holistic, therapeutic-agnostic platform technology for the administration, activation of propulsion, and manufacture of a pharmaceutical composition configured to physically penetrate and/or embed an active ingredient (e.g., a pharmaceutical agent) within a biological tissue or barrier, such as the blood brain barrier or a gastrointestinal barrier, for enhanced drug absorption. The physical penetration of the pharmaceutical agent across the biological barrier is promoted by the evolution of a propulsion gas driving the agent into the tissue thereby increasing the penetration, retention time, and absorption of the pharmaceutical agent. The pharmaceutical compositions and methods can be used to deliver a pharmaceutical agent across a plurality of biological barriers and tissues.

Described herein are compositions for propulsion-enabled API entities, where the entities are capable of motion due to a propulsion gas being emitted to propel one or more pharmaceutical agents(s) towards and into a biological barrier such that the active ingredient(s) becomes embedded in the biological barrier. Also provided are methods of administering a therapeutically effective amount of propulsion enabled entities to treat a disease with a focus on nasal, oral and sublingual administration.

### Pharmaceutical Compositions

In some embodiments of the invention, the API neat particles and API containing particles can be prepared using manufacturing techniques such as spray drying, lyophilization, freeze-lyophilization, freeze spray drying, solvent evaporation or any other regulatory approved manufacturing technique as is known by those skilled in the art. The active pharmaceutical ingredient may be encapsulated such as within a liposome. API containing particles may contain polymers, lipids, carbohydrates, or a combination of these or other regulatory-agency approved components. Neat API particle sizes and API containing particle sizes can be varied by varying the synthesis conditions, as is known by those skilled in the art, in order to tune the release properties of the particles based on the active ingredient to be delivered, the required release location (e.g. upper or lower nasal cavity) and the release profile.

In some embodiments of the invention, the propulsion agent (e.g. a carbonate or bicarbonate) may be incorporated within the pharmaceutical composition in one of the following locations, or a combination of more than one: 1. Admixed with the neat API or API containing particles 2. within the API containing particle as a result of co-spray drying, co-lyophilization or any other form of co-processing with the active ingredient and any other excipient 3. incorporation within granules or aggregates of neat API particles or API containing particles along with any other excipient (intragranular) 4. incorporated after the granulation or aggregation of API particles or API containing particles (intergranular). Admixing of the propulsion agent with API entities such as API neat particles, API containing particles or API granules or aggregates serves to coat the surface of these API entities via a mechanofusing mechanism where particles adhere due to surface forces, which are especially magnified for particles on the micron, sub-millimeter and millimeter scale. The benefit of this drug delivery platform is that it is particle and powder agnostic.

Examples of the propulsion agent component that may be used in the present invention include carbon dioxide, nitrogen, nitrogen dioxide, hydrogen, oxygen and water vapor evolving compounds. Carbon dioxide evolving compounds such as carbonates or bicarbonates, may include carbonates that are classified as soluble or insoluble, such as magnesium carbonate, iron (II) carbonate, calcium carbonate, sodium carbonate, potassium carbonate, sodium hydrogen carbonate or potassium hydrogen carbonate, sodium bicarbonate, potassium bicarbonate, magnesium bicarbonate, calcium bicarbonate, ammonium bicarbonate, carbonic acid, or the like. Nitrogen dioxide and nitrogen producing compounds include nitrates or nitrites.

In some embodiments of the invention, propulsion gas production occurs when the propulsion agent (e.g. a carbonate or bicarbonate) comes into contact with the activation agent which can be water or an acidic solution based on the properties of the propulsion agent (e.g. for a soluble propulsion agent it may be water, whereas for insoluble one it may be an acidic solution).

In some embodiments, the activation agent may be water. In some embodiments of the invention, the activation agent may be an acidic solution of a pH below about 7. In some embodiments of the invention, the activation agent may be an organic or inorganic acid solution or mixtures thereof. Examples of in the present invention include, e.g. hydrochloric acid, citric acid or its salts such as sodium citrate or calcium citrate; malic acid, tartaric acid, succinic acid, fumaric acid, maleic acid, or their salts; ascorbic acid or its salts such as sodium or calcium ascorbate; glycine, sarcosine, alanine, taurine, glutamic acid; lactic acid or its salts; acetic acid or its salts; oxalic acid or its salts; and the like. An acidic solution activation agent would also serve to solubilize or loosen one or more biological structures, e.g., mucus, to allow for further penetration of the pharmaceutical across the barrier yet of only a small quantity to prevent any potential irritation e.g. microliter. In some embodiments, the activation agent may be a solution of pH in the range of about 1 to about 14. In some embodiments, the activation agent may react with the propulsion agent such that a unidirectional release of a chemical in the form of a gas will occur from the exposed region of the particles and propulsion into the barrier will occur.

In some embodiments of the invention, the activation agent may be separately administered from the pharmaceutical composition. For example, for nasal administration, a first spray containing the pharmaceutical composition containing the API entity and the propulsion agent (e.g. carbonate) would be administered; followed by a second spray administration of the activation agent (e.g. an acidic solution) into the same location of the nasal cavity. Upon contact of the propulsion and activation agents, a gas is released from the exposed portion of the API entity propelling it to embed into the nasal tissue. Hence upon being administered into the nasal cavity, an API entity (e.g. in powder form) that is coated with the propulsion agent (e.g. Magnesium carbonate) would adhere to the nasal lining, with one portion attached to the lining and the other exposed to the nasal cavity. Upon administering the second spray of the activation agent (e.g. an acetic acid solution), the activation agent would come into contact with the propulsion agent resulting in the asymmetric release of a propulsion gas (e.g. carbon dioxide) from the exposed portion of the coated API entity. Gas release from the coating of the API entities results in a transfer of momentum to the particles propelling and moving them into the tissue.

In some embodiments of the invention, the activation agent is a fluid(s) or moisture already existing within the body such as gastric fluids, saliva and moisture within mucosal membranes that do not require separate administration.

In some embodiments of the invention, excipients can be incorporated into the pharmaceutical compositions and where they can be: 1. physically mixed with the API in solid form 2. co-lyophilized, co-spray-dried or co-processed with the active ingredient 3. forming a matrix in which the drug is dispersed, such as in the case of the polymeric microspheres; 4. forming a protective shell around the active ingredient such as in microcapsules and liposomes.

### Nasal Administration

The pharmaceutical compositions of the disclosure include those formulated for nasal administration (“nasal dosage forms”). Nasal dosage forms can be, for example, in the form of sprays, emulsions, suspensions, and the like, which contain the active ingredient(s) in a mixture with non-toxic pharmaceutically acceptable excipients. These excipients may be, for example, inert diluents or fillers; granulating and disintegrating agents; binding agents; and lubricating agents, glidants, and antiadhesives. Other pharmaceutically acceptable excipients can be colorants, flavoring agents, plasticizers, humectants, buffering agents, and the like.

In some embodiments of the invention, administration of the pharmaceutical composition nasally may be used for nose-to-brain delivery of the pharmaceutical agent(s), the systemic delivery of the pharmaceutical agent(s) or delivery of the pharmaceutical agent(s) to non-brain target organs.

In some embodiments, the pharmaceutical composition for nasal administration may be formulated to attach to the lining within the nose, in which, an activation agent is administered into the nasal cavity to activate the propulsion coating and emit the propulsion gas.

In some embodiments of the invention, the pharmaceutical composition for nasal administration may be in the form of an intranasal powder. Where intranasal powders tend to offer improved chemical stability for the API. Alternatively, in some embodiments the pharmaceutical composition may be in the form of an intranasal liquid spray. In some embodiments of the invention, in which the particles are in a suspension. The suspension may have one or more buffers or pharmaceutically acceptable excipients. For example, a pharmaceutical composition that is in the form of an intranasal spray may have particles in a pH neutral suspension, in which the adherence of the propulsion coating to the particle is not affected by one or more chemical or physical properties of the suspension, and in which the propulsion coating is a compound that is only activated at low acidic pH (e.g. an insoluble carbonate).

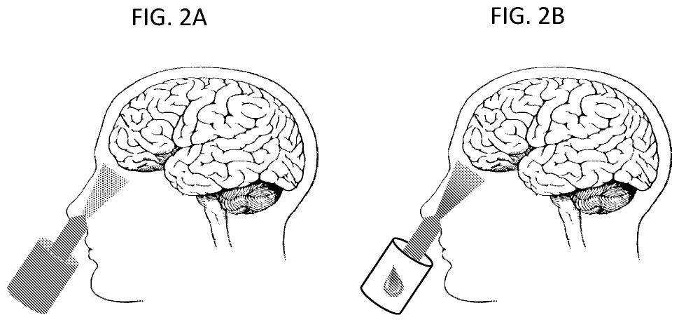

In some embodiments, the pharmaceutical composition is formulated to generate propulsive motion by the administration of an activation agent. After the administration of the API entities with propulsive capability, propulsive motion will be triggered by the administration of an intranasal spray of either just water in the case of a soluble carbonate or an acidic solution in the case of either a soluble or insoluble carbonate. The pharmaceutical composition may have a soluble compound (e.g. soluble carbonate salt) as the propulsion agent, in which water may be the activation agent. In some embodiments, the pharmaceutical composition may have an insoluble compound (e.g. insoluble carbonate salt) in which an acidic solution may be the activation agent. In some embodiments, an acidic component (e.g. acid in powder form) may be included in the pharmaceutical composition dosage form (e.g. powder) such that water can be the activation agent regardless of the solubility properties of the propulsion agent. In some embodiments, an acidic spray used for both soluble and insoluble propulsion agents would also assist in the loosening and dissolution of the mucus to allow further penetration of the particles across the barrier. The secondary spray with an acidic pH will also result in a faster release of gas for a soluble propulsion agent than water, and hence a larger penetration force resulting in more effective delivery. Upon exposure to this secondary spray, a unidirectional release of carbon dioxide will occur from the exposed region of the granules and propulsion into the barrier will occur. FIG. 2.

In some embodiments, the pharmaceutical composition may have calcium containing propulsion agents (e.g. calcium bicarbonate), in which the presence of calcium ions causes increased activity of the nasal cilia and the clearance of the formulation from the cavity. Hence in general calcium containing propulsion agents may be avoided or a simultaneous incorporation of calcium collecting excipient may be incorporated in the pharmaceutical composition to remove the excess calcium ions. In some embodiments, the pharmaceutical composition may have propulsion agents that do not contain calcium.

In some embodiments of the invention, API entities with propulsive capability, that possess the ability to undergo powered motion when exposed to either water or an acidic activation agent solution, will be administered via the nasal cavity. After this first administration step, the particles will have been attached to the nasal lining. Once adhered to the nasal lining, the activating agent spray will be sprayed into the nose triggering the release of gas asymmetrically from the API entity's exposed portion leading to the motion of the API entities to penetrate or embed further within the nasal lining. This will result in the increased retention time, absorption and hence penetration of the contained active ingredient for increased bioavailability. Schematic of the process in FIG. 2.

It should be noted that the particular identity of the propulsion agent and activation agent will be selected based on the properties of the active ingredient to be delivered as well as the overall formulation and pharmaceutical composition. For example, acid sensitive API's may be formulated with a soluble propulsion agent and utilize water as the activation agent.

In some embodiments of the invention, the pharmaceutical composition for nasal administration may be in the form of an intranasal powder. Intranasal powder formulations, where for humans a typical maximum of 25-50 mg [International Journal of Pharmaceutics 561 (2019) 47-65] is delivered in a single administration in a single nostril, the weight of excipients is ideally minimized and if possible eliminated, yet it is common and sometimes compulsory to add various excipients for a number of purposes including accurate and uniform dosing for high potency drugs (<1-5 mg per unit dose) or solubility enhancement of the drug in the mucosa, mucoadhesion to increase powder retention time, or absorption enhancers which may include enzyme inhibitors. Common examples of excipients include fillers or diluents such as microcrystalline cellulose (MCC), colloidal MCC, lactose and mannitol where fillers can either be soluble or insoluble where these properties may be used to enhance the solubility of the drug based on its properties. A single or multiple fillers may be use at different relative amounts by weight. Common examples of mucoadhesive agents include cellulose derivatives like hydroxypropyl cellulose (HPC), hydroxypropyl methyl cellulose (HPMC), hydroxypropyl ethyl cellulose (HEC), sodium carboxymethylcellulose, carboxymethyl cellulose (CMC), Pectin, as well as Chitosans and its derivatives such as Chitosan Carboxymethyl Chitosan. Common examples of absorption enhancers include several substances, differing for their mechanisms of action, are: surfactants (e.g. sodium laurylsulfate, saponin, polisorbate 80, laureth-9); bile salts and derivatives (e.g. sodium glycocholate, sodium taurocholate, sodium deoxycholate); fatty acids and derivatives (e.g. sodium caprylate, sodium laurate, oleic acid); phospholipids (e.g. lysophosphatidylcholine, idecanoylphosphatidylcholine); glycyrrhetinic acid derivatives (e.g. carbenoxolone, glycyrrhizinate); chelating agents (e.g. ethylenediaminetetraacetic acid, salicylates); cyclodextrins (α-, β-, γ-cyclodextrins and their derivatives); cationic compounds (e.g. chitosan and derivatives, poly-L-arginine, poly-L-lysine). Considerations that one skilled in the art makes for the selection of specific excipients include compatibility with that physiochemical properties of the drug to be delivered, for example its oxidation sensitivity when mixed with various excipients. In some embodiments of the invention, we add a gas producing propulsion agent to the formulation.

In some embodiments of the invention, the pharmaceutical composition for nasal administration may be in the form of an intranasal powder comprising:

1. One or more active pharmaceutical ingredients or API entities in an amount of up to about 95% by weight.
2. One or more fillers such as those listed above about 0 to about 90% by weight.
3. One or more mucoadhesive agents about 0 to about 80% by weight and where they may be co-formulated with the active ingredient in particles.
4. One or more gas producing propulsion agent(s) such as a carbonate or bicarbonate from about 0.5% to about 95% by weight, optimally in the range of 5% to 50% by weight.
5. One or more acid(s) in powder form in the range of 0% to 85% by weight, to allow the use of water, as the triggering agent in the case of insoluble carbonates; or to increase the rate of release of gas in the case of a soluble carbonate.
6. Absorption enhancers such as those listed above from about 0% to 50% by weight
7. Degradation enzyme inhibitors from about 0 to 25% by weight.
wherein the amounts by weight are based on the total weight of the composition.

In its simplest form the pharmaceutical composition would contain a minimum of two components, the active pharmaceutical ingredient and the gas producing agent.

In some embodiments of the invention, following the administration of the nasal powder, a gas release triggering nasal spray containing the activation agent will be administered. The amount of time between the administration of the pharmaceutical composition and the activation agent stray may range from immediately, namely fractions of second where the second spray may be automatically administered from the same device or up to hours after the first administration of the powder. Typically nasal formulations are targeted to adhere within the nasal cavity up to 30 minutes prior to ciliary clearance. Hence practically the triggering spray maybe released within fractions of a second to 30 minutes from the time of the administration of the powder. This may be done immediately after the nasal powder, in fractions of a second, from the same device without removing it from the nostril or may also be done from a second device. In some embodiments, the time between the administration of the pharmaceutical composition and the activation agent may range from fractions of a second, to seconds, to minutes to hours. The pH of the nasal spray may range anywhere on the pH scale depending on the identity of the gas releasing activation agent where for insoluble carbonates for example lower pH acidic sprays (pH 0-7) would be best, optimally between a pH or about 3 to about 6. For powder formulations where a dry acid powder component is incorporated within the formulation, a neutral activation agent such as water may be used.

In some embodiments, more than one administration of the activation agent is administered, wherein administrations following the initial administration of an activation agent occurs at a time ranging from fractions of a second to more than an hour.

In some embodiments of the invention, the pharmaceutical composition and activation agent nasal spray of the embodiments may be delivered by any one of the known in the art nasal devices, such as pressurised devices, dry powder sprayers or bi-directional nasal devices.

In some embodiments, for propulsion agents that are insoluble (e.g. some carbonates), they may be administered as an intranasal spray where the particles are in a pH neutral suspension, so long as the adherence of the salt to the particles is not affected by their presence as a suspension. Intranasal powder tends to offer improved chemical stability for the API.

Nasal spray volumes typically range from 25 to 200 μL with dose volumes of sprays on the market normally between 50 μL and 100 μL. [Journal of Pharmaceutical Investigation (2020) 50:251-259]. The local pH value inside the nasal cavity will have a direct effect on the rate and extent of absorption of drugs; the optimal range for pH value of the nasal spray formulation is suggested to be between 4.5 and 6.5 with some nasal sprays on the market with a pH as low as 3.5. In some embodiments of the invention, the nasal spray volumes with be in the range from about 25 to about 200 μL. In some embodiments of the invention, the propulsion, gas release triggering spray can have a range of pH values within the safety limits allowed by regulatory agencies from a pH 0 to 14. In some embodiments of the invention, the optimal range for pH value of the activation agent nasal spray may be between 3.5 and 6.

### Oral Administration

The pharmaceutical compositions of the disclosure include those formulated for oral administration (“oral dosage forms”). Oral dosage forms can be, for example, in the form of tablets, capsules, a liquid solution or suspension, a powder, or liquid or solid crystals, which contain an active ingredient(s) (e.g., a pharmaceutical agent) in a mixture with non-toxic pharmaceutically acceptable excipients. These excipients may be, for example, inert diluents or fillers; granulating and disintegrating agents; binding agents; and lubricating agents, glidants, and antiadhesives. Other pharmaceutically acceptable excipients can be colorants, flavoring agents, plasticizers, humectants, buffering agents, and the like.

Pharmaceutical compositions for oral administration may also be prepared as a chewable tablet, as a hard gelatin capsule (e.g., in which the active ingredient (e.g., a pharmaceutical agent) may be, e.g., mixed with an inert solid diluent), or as a soft gelatin capsule (e.g., in which the active ingredient (e.g., a pharmaceutical agent) is mixed with water or an oil medium, for example, peanut oil, liquid paraffin, or olive oil). Powders, granulates, and pellets may be prepared using the ingredients mentioned above under tablets and capsules in a conventional manner using, e.g., a mixer, a fluid bed apparatus, or a spray drying equipment.

In some embodiments, the pharmaceutical composition may also include a coating such as an enteric coating.

In some embodiments the pharmaceutical composition may be composed based on a targeted site of delivery. In some embodiments, the targeted site of delivery may be any biological structure involved in the gastrointestinal tract, e.g., oral cavity, buccal cavity, esophagus, stomach, small intestine, large intestine, or the like. In some embodiments, the targeted site of delivery may be sublingual.

In some embodiments, a pharmaceutical composition of the disclosure may be formulated for delivery of a pharmaceutical agent to a target site based on specific conditions at the target site. For example, a pharmaceutical composition can be formulated to accommodate delivery to a site having a known pH. For example, a pharmaceutical composition that is sensitive to acidic conditions, in which the target location of delivery will be in the small intestine or in the abdomen and taken with food, can be formulated using a water-soluble gas producing compound, such as a bicarbonate or sodium carbonate. Alternatively, a pharmaceutical composition that has a non-water-soluble carbon dioxide producing salt, such as calcium carbonate or magnesium carbonate, may be used with the incorporation of a weak acid, such as an organic acid, within the formulation to assist in dissolution. In some embodiments, a pharmaceutical composition that is not extremely sensitive to weak acids, may be formulated with an additional acid for a more rapid release of gas for example from a non-water-soluble carbonate, faster motion of the particles and penetration with a higher level of force within the intestinal lining.

In some embodiments, the pharmaceutical composition may have a targeted site of delivery within sites of largely neutral or only slightly acidic regions, such as within the small intestines. In this embodiment, a water-soluble gas producing salt can be used, such as a bicarbonate with an enteric drug product coating or capsule. Alternatively, an insoluble salt may be used for release in these locations, in which a weak acid powder may be incorporated in small amounts in the pharmaceutical composition to facilitate localized low pH only in the vicinity of the gas producing propulsion agent to induce dissolution and rapid gas production for the increased penetration, improved embedding, and increased retention time in the gastrointestinal barrier. In some embodiments, acid for localized decrease of pH may also be incorporated when using water-soluble gas producing salts as well for localized lowering of the pH and more rapid release of gas (e.g. CO₂) and hence more rapid movement and improved penetration again. An acid can be selected that produces no adverse effect on the active ingredient(s).

In some embodiments of the invention, the pharmaceutical composition, formulated with active pharmaceutical ingredients and excipients, admixed with or coated with a gas releasing propulsion agent (e.g. carbonate), is administered orally, wherein the activation agent(s) are gastric fluids already present within the gastrointestinal tract. Evolution of the propulsion gas is triggered by contact with these fluids which promotes embedding or penetration of all or a part of the pharmaceutical composition in a biological tissue or a biological barrier.

In some embodiments of the invention, the pharmaceutical compositions for oral administration (oral dosage forms) may comprise tablets, capsules, a liquid solution or suspension, a powder, or liquid or solid crystals, which contain an active ingredient(s), propulsion agent(s) comprising a component capable of evolving a propulsion gas a propulsion, in a mixture with non-toxic pharmaceutically acceptable excipients.

In some embodiments of the invention, the pharmaceutical composition is an oral tablet of a powder comprising: one or more active pharmaceutical ingredients, one or more diluents (e.g. microcrystalline cellulose, lactose, mannitol, starch and the like), one or more disintegrants or super disintegrants (e.g. sodium starch glycolate, croscarmellose sodium, crospovidone and the like) to ensure the breakup of a tablet formulation, one or more lubricants, anti-adherents and glidants (e.g. magnesium stearate, stearic acid, and the like), one or more gas producing propulsion agents (e.g. carbonate, bicarbonate and the like), potentially one or more acids in powder form to lower the local pH surrounding the tablet increasing the rate of gas production in the case of both soluble or insoluble gas producing propulsion agents, potentially one or more degradation enzyme inhibitors and potentially one or more permeation enhancers.

In some embodiments of the invention, an oral dosage form may be formulated as an oral tablet to be swallowed, a sublingual tablet or the powder maybe loaded into an oral capsule. The oral dosage forms containing the propulsion enabled particles for delivery across the GI barrier comprises:

1. One or more active pharmaceutical ingredients in an amount of up to about 95% by weight.
2. One or more diluents to increase bulk volume for handling such as those listed like microcrystalline cellulose, lactose, mannitol, starch and their derivatives at about 0 to 90% by weight with most diluents between about 20% to about 90% by weight.
3. One or more disintegrants to ensure the breakup of a tablet formulation at between about 0% and 25% by weight with most disintegrants within about 0.5% and about 15%.
4. One or more lubricants, which can act as anti-adherents and glidants, lubricants include magnesium stearate, stearic acid, hydrogenated vegetable oil and the like at between about 0% and about 3% by weight.
5. One or more anti-adherents and glidants at between about 0% and about 3%.
6. A gas producing propulsion agent such as a carbonate or bicarbonate from about 0.5% to about 95% by weight, optimally in the range of about 5% to about 50% by weight. This gas producing propulsion agent also simultaneously acts as a disintegrant and hence may serve as a dual-purpose component with no traditional disintegrants such as sodium starch glycolate, croscarmellose sodium, crospovidone and the like needed.
7. An acid in powder form, to serve as a local activation agent in the case of insoluble carbonates; or to increase the rate of release of gas in the case of a soluble carbonate, at about 0% to about 50% by weight.
8. One or more enzyme inhibitors from about 0% to about 15% by weight.
9. One or more absorption/permeation enhancers from about 0% to about 15% by weight. wherein the amounts by weight are based on the total weight of the composition. with the percentages of the different components adding up to about 100%.

In some embodiments, these powder components maybe compressed into tablets for oral or sublingual administration. For acid sensitive therapeutics, such as proteins and biologics, and in cases where drug absorption occurs in the small intestine, tablet film coating with a protective enteric coating may be conducted such that the therapeutic is not exposed to the sometimes very low pH of the stomach environment (pH 1-3). In these cases, a water-soluble gas producing propulsion agent is used.

Alternately, in some embodiments, a weak organic acid in powder form may also be incorporated at a low weight percentage (5-25%) to achieve a slight reduction in the local pH upon the disintegration of the tablet hence increasing the rate of gas production and therefore the speed and force with which the active pharmaceutical ingredient is embedded within the lining of the GI tract. Hence, while the nasally administered propulsion-enabled formulation utilizes the administration of a second acidic or neutral spray as the activation agent for the production of the propulsion gas, for orally administered propulsion enabled formulations, hydration and propulsion is enabled by bodily fluids within the GI tract or moisture existing in the mouth in the case of sublingual tablets. The propulsion-enabled formulation may also be administered by loading into enteric capsules or non-enteric capsule based on the location of absorption of a particular therapeutic.

In some embodiments of the invention, an oral tablet may be enterically coated or the powder may be loaded into a capsule or an enteric capsule.

In some embodiments of the invention, the pharmaceutical composition, formulated with active pharmaceutical ingredients, excipients such as diluents and a gas releasing propulsion agent, is administered sublingually, wherein the activation agent(s) are moisture already present under the tongue. Evolution of the propulsion gas is triggered by contact with this moisture (e.g. saliva) which promotes embedding or penetration of all or a part of the pharmaceutical composition in a biological tissue or a biological barrier.

In some embodiments of the invention, the sublingual dosage forms of the pharmaceutical composition can be in the form of powders, sprays, tablets, films and the like, which contain the active ingredient(s) in a mixture with non-toxic pharmaceutically acceptable excipients

Exemplary routes of administration of the pharmaceutical compositions include nasal, oral (e.g. gastrointestinal), sublingual, rectal, ocular administration, transdermal administration, and the like. In some embodiments, a pharmaceutical is provided by mucosal administration, rectal administration, ocular administration, vaginal administration, transdermal administration, and the like.

### Indications and Pharmaceutical Agents

While this invention represents a therapeutic-agnotistic platform, illustrative examples of drugs that can utilize the present invention for nasal delivery include sumatriptan succinate, zolmitriptan salts, naratriptan, riza-triptan, almotriptan, eletriptan, frovatriptan, bupivacaine, fibroblast growth factor, cephalexin, lidocaine, clobazame, midazolam, alprazolam, diazepine, lorazepam, dexmedeto-65 midine, monosialoganglioside, cocaine, insulin, glucagon, oxytocin, fentanyl, sulfentanil, diamorphine, ketamine, apo-morphine, buprenorphine, morphine sulphate, oxycodone hydrochloride, butorphanol, NSAIDs, paracetamol, benzo-diazepines, dopamine, pramipexole, rasagiline, rogitine, ondansetron, granisetron, metoclopramide, naloxone, naltrexone, atropine, adrenaline, cannabis active compounds, epinephrine, isosorbide dinitrate, obitoxine, dexmedetomi-dine, metochlorpramide, L-dopa, nicotine, sildenafil, nafarelin, dobutarnine, phenylephrine, tramazoline, xylometazo-line, tramadol, methacholine, ipratropium, scopolamine, propranolol, verapamil, hydralazine, nitroglycerin, clofilium tosylatecannabis active compounds and pharmaceutically acceptable salts, isomers, and mixtures thereof.

These drugs span a wide range of indications including use in common cold treatment, anti-addiction agents, anti-infective agents, analgesics, anaesthetics, antarthritics, anti-allergy agents, antiasthmatic agents, anticonvulsants, anti-depressants, antidiabetic agents, anti-diuretics, anti-emetics, antihistamines, anti-hypertensive agents, anti-inflammatory agents, antimigraine preparations, anti-motion sickness preparations, antinauseants, antineoplastics, anti-obesity, antiosteoporosis, anti-Parkinsonism drugs, antipru-ritics, antipsychotics, antipyretics, anticholinergics, benzodiazepine antagonists, bone stimulating agents, central nervous system stimulants, hormones, hypnotics, immuno-suppressives, prostaglandins, proteins, peptides, polypep-tides and other macromolecules, psychostimulants, com-pounds for use in rhinitis treatment, compounds for use in sexual hypofunction treatment, sedatives, compounds for use in treatment of known or suspected opioid overdose, tranquilizers and vitamins, probiotics, natural ingredients, peptide or protein therapeutic agents such as cytokines, hormones, clotting factors, vaccines, monoclonal antibodies, amino acids, or any combination thereof.

In some embodiments of the invention, and in particular those that are nasally administered, have the potential to have a significant impact on nose-to-brain delivery and potentially delivery across the blood brain barrier. Hence have a significant impact on the treatment of brain, CNS and neurological disorders, or complications resulting therefrom. Non-limiting examples of neurological disorders include multiple sclerosis (MS), ischemia, amyotrophic lateral sclerosis (ALS), Parkinson's disease (PD), Alzheimer's disease (AD), autism, Rett syndrome, Fragile X syndrome, Angelman syndrome, cerebral palsy, Down syndrome, Dravet syndrome, epilepsy schizophrenia, bipolar disorder, anxiety disorder, major depressive disorder (MDD), and the like.

Additional illustrative examples of drugs that are suitable for oral administration or sublingual administration utilizing the present invention include antibacterial/anti-infective agents, such as ciprofloxacin, cefuroxime, cefatrizine, cefpodoxime, clarithromycin, loracarbef, azithromycin, cefixime, cefadroxil, amoxycillin, and the like; antivirals, such as acyclovir; cardiovascular agents, such as diltiazem, captopril and the like; non-steroidal anti-inflammatory agents, such as etodolac, ketorolac, and the like; anti-ulcer agents, such as ranitidine, famotidine, and the like; drugs for respiratory diseases, such as fexofenadine, pseudoephe-drine, phenylpropanolamine, dextromethorphan, chlorphe-30 niramine, and the like; dopaminergic agents, such as bro-mocriptine; immunosuppressants, such as cyclosporin; skeletal muscle relaxants, such as baclofen; anti-gout agents, such as allopurinol; and the like.

While this invention represents a therapeutic-agnostic platform, illustrative examples of drugs that can utilize the present invention for drug delivery include therapeutics which can be classified as small molecules, macromolecules (e.g. a peptide, protein or biologic), BCS I class drugs, BCS II class drug, BCS III class drug and BCS VI class drugs.

### Manufacturing Process and Unit Operations:

The manufacturing process of the disclosure includes producing a pharmaceutical composition that has an active ingredient(s), one or more pharmaceutically acceptable excipients and the gas releasing propulsion agent. The active pharmaceutical ingredient(s) (API) within a composition may be prepared as a neat powder or incorporated within particles that can include polymers, lipids, carbohydrates, or a combination of these or other components approved by regulatory agencies for use in pharmaceutical compositions. Neat API particles, API containing and API encapsulating particles (e.g. liposomes) may be prepared using manufacturing techniques such as spray drying, lyophilization, freeze-lyophilization, freeze spray drying, solvent evaporation or any other regulatory approved manufacturing technique known to those skilled in the art. Particle sizes can be varied by varying the synthesis conditions, as known to those skilled in the art; in order to tune the release properties of the particles based on the active ingredient to be delivered, the required release profile and the location of delivery.

In some embodiments, the neat API or API containing particles, referred to simply as API powders, can be blended directly with any excipients including the gas releasing propulsion agent and used as such; or granulated/agglomerated with excipients with or without the gas releasing propulsion agent incorporated within the granule. As such, in some embodiments, the gas releasing propulsion agent may be incorporated within the powder formulation in one of four locations or combinations thereof:

1. Admixed with or coating the active pharmaceutical ingredient, where coating of the API can occur in a simple blending process via a mechanofusing mechanism where particles adhere due to each other as a result of surface forces which are especially magnified for particles on the micron, sub-millimeter and millimeter scale.
2. Within the particles containing the active pharmaceutical ingredient as a result of co-processing, such as co-spray drying with the active pharmaceutical ingredient and any other excipient
3. Incorporated within granules or aggregates of neat active pharmaceutical ingredient particles or active pharmaceutical ingredient-containing particles along with any other excipient
4. Incorporated intergranular or coating the granules or aggregates.
In terms of combinations of the four locations, an example would be granules that incorporate the gas releasing propulsion agent, that are also coated with it.

In some embodiments, the flow properties, the manufacturability and the content uniformity of the resultant dosage form, especially with low dose active ingredients, may be improved with granulation or aggregation. Granulation or aggregation also prevents the segregation of different powders within the formulation during processing. Additionally larger particles would have a reduced risk of lung inhalation in the case of nasal administration, which is especially the case if particles are below 10 microns. In some embodiments, dry granulation or wet granulation approaches may be used.

Dry granulation or agglomeration is especially suited for moisture sensitive API's. In some embodiments, dry granulation methods, such as roller compaction, may be used. In some embodiments, the API powder is first blended with pre-granulation excipients such as fillers, disintegrants (e.g. soluble chloride salts such as potassium or sodium chloride), binders, lubricants and potentially the gas releasing propulsion agent which can also serve a dual purpose as a disintegrant or other excipients as described herein. In some embodiments, other excipients such as degradation enzyme inhibitors, permeation enhancers, mucoadhesive agents, powder acids, or other excipients may also be incorporated into the blend.

After this blending step, in some embodiments, a dry granulation will be carried out most likely using roller compaction. In some embodiments, granules can be coated with the gas releasing propulsion agent via blending or mechanical agitation to allow the mechanofusing of the smaller propulsion agent particles with the surface of the larger granule. In some embodiments, after coating the active ingredient granules with the propulsion agent using the mechanofusing mechanism, where the propulsion agent has a grain size that is smaller than the size of the active ingredient granules, the mixture will be sieved through a sieve that will allow excess gas-producing propulsion agent, to pass through the sieve, while trapping the active ingredient containing granules that are coated. In some embodiments, after the sieving step, the coated granules may be blended with other excipients for the optimal delivery of the active ingredient based on the biological barrier of interest. This formulation may then be packaged in a delivery device.

In some embodiments, granule coating utilizing other methods such as pan film coating may also be used. In some embodiments, the granule or granule coating formulation may also incorporate an acid for the faster release of gas and hence faster propulsion. In some embodiments, after processing within a roller compactor to produce granules, the blend can be further lubricated along with a compression aid such as a diluent like MCC, for the production of oral or sublingual tablets or may be load it into capsules without the additional lubrication or packaged into a delivery device.

In some embodiments, such as in the production of intranasal powder, the need would be for loosely or weakly compacted granules with an emphasis on rapid disintegration upon contact with the nasal lining. Roller compaction, as described above, with a low force can be used to allow for the facile disintegration of granules into particles upon administration within the naval cavity.

Alternatively, in some embodiments, instead of low force roller compaction an agglomeration method may be used. As such agglomeration based on simple mechanical agitation that yield more rapidly disintegrating agglomerates may be used. This would be well suited for nasal powder manufacture. In some embodiments, agglomeration may be accomplished via mechanical agitation of the components. Agitation may be done using the vibration of a pile of analytical sieves via a mechanofusing mechanism, with the size selection of the agglomerates determined by a sieving step that may follow. In some embodiments, the gas propulsion agent may be incorporated into the aggregates and/or used to coat them. In some embodiments, this pharmaceutical composition is then compacted into tablets, loaded into capsules or packaged in a nasal delivery device.

In some embodiments, wet granulation and agglomeration approaches may also be used as are well known to those skilled in the art. Agglomeration methods include Tumbling Agglomeration, Steam-jet agglomeration, Spray drying agglomeration, Fluid bed agglomeration or granulation among others.

The manufacture of the water or acidic solution spray that serves as the activation agent for nasal administration to trigger the release of the propulsion gas will be done utilizing the established GMP manufacture method of preparing liquid nasal sprays with absence of any steps or complications related to the inclusion of an active pharmaceutical ingredient (API).

This liquid spray that is in the second step in the administration of the nasal dosage form may be contained either within the same device as the API in the first administration in a separate compartment or in an additional device. The device design will be addressed in another patent.

In some embodiments, where a granulation step is not present, each active ingredient particle may be coated with or adhered to the propulsion coating, e.g., gas producing salt. In some embodiments, this may be accomplished via direct blending. In this embodiment, after blending to coat the active ingredient particle, sieving would be used to remove excess unadhered powders.

In some embodiments of the invention, a manufacturing process for the production of a powder of composition with an active pharmaceutical ingredient(s) coated with the gas releasing propulsion agent via simple mechanofusion including:
iii) Sieving each individual component of the formulation to ensure the removal of any clumping
iv) Blending of the components which includes the active pharmaceutical ingredient(s) and the gas producing propulsion agent, and can include diluents, disintegrants and lubricants if needed.
   wherein blending of the active pharmaceutical ingredient(s) and the gas producing propulsion agent leads to the mechanofusing of the different particles, where they adhere due to surface forces which are especially magnified for particles on the micron, sub-millimeter and millimeter scale.

In some embodiments of the invention, a manufacturing process for the production of a powder composition incorporating dry granulation or agglomeration for nasal, oral or sublingual administration, where active pharmaceutical ingredient(s) containing granules both contain and are coated with the gas releasing propulsion agent comprises:
i) Sieving each individual component to ensure the removal of any clumping
ii) Blending of the pre-granulation components which includes the active pharmaceutical ingredient(s) and gas producing propulsion agent, and can include diluents, and other excipients such as disintegrants and lubricants as needed.
iii) Dry Granulation or Agglomeration (e.g. roller compaction, mechanical agglomeration using a sieve shaker or the like)
iv) Coating granules with gas producing propulsion agent. This may be done using simple blending via a mechanofusing mechanism or via other methods such as film coating. Optional additional excipients may be added here.
v) Sieving to remove any excess gas producing propulsion agent (with this being an optional step)
vi) Blending with any other excipients (optional step and may be done to assist in compaction into tablets for oral or sublingual administration)

The resulting powder can be: i) loaded into a delivery device for nasal administration, ii) loaded into a capsule that is either enteric or non enteric for oral administration, iii) compacted into tablets that can either be coated with an enteric or non-enteric coating, or not coated for oral administration or iv) compacted into tablets for sublingual administration.

In some embodiments of the invention, a manufacturing process to produce active pharmaceutical ingredient containing entity granule that is just coated with the gas releasing propulsion agent process comprises: To produce a API containing entity grow granule that is both coated with and contains the gas releasing propulsion agent:
i) Sieving each individual component to ensure the removal of any clumping
ii) Blending of the pre-granulation components which includes the active pharmaceutical ingredient(s) and can include diluents, disintegrants and lubricants if needed.
iii) Dry Granulation or Agglomeration (e.g. roller compaction, mechanical agglomeration using a sieve shaker or the like)
iv) Coating granules with gas producing propulsion agent. This may be done using simple blending via a mechanofusing mechanism or via other methods such as film coating. Optional additional excipients may be added here.
v) Sieving to remove any excess gas producing propulsion agent (with this being an optional step)
vi) Blending with any other excipients (optional step and may be done to assist in compaction into tablets for oral or sublingual administration)

In some embodiments of the invention, a manufacturing process as described above where wet granulation is used rather than dry granulation.

In some embodiments of the invention, a process for the loading of intranasal powders or sprays into a delivery device is used.

In some embodiments of the invention, a process for the production of oral or sublingual tablets of the pharmaceutical compositions by utilizing tablet compaction.

In some embodiments of the invention, a process for the coating of tablets with enteric or non-enteric capsules utilizing a film coating process.

These embodiments of manufacturing approaches and unit operations, represent non-limiting approaches that do not exclude others.

### EXAMPLES

Non-limiting examples to embody this invention are listed below.

While the BCS has classification is typically used for orally administered compounds it will be used here as a way to classify drugs whether administered orally, sublingually or nasally for their water solubility and permeability. Presented are examples utilizing more than one class encompassing a range of indications.

### Example 1: Model Small Molecule Permeation

The first non-limiting example to embody this invention is one in which a small molecule was selected to test the permeation and transport enhancement due to propulsion. Propulsion is due to the production of gas from a formulation containing a propulsion agent after contact with an activation agent. The molecule Erioglaucine (C₃₇H₃₄Na₂N₂O₉S₃; CAS number: 3844-45-9 and a molecular weight of 792.85 g/mol) which has a blue color absorbing in the red range on the color spectrum will be used as the model molecule with colorimetry used to quantify permeation.

### Powder Formulation Using an Insoluble Propulsion Agent

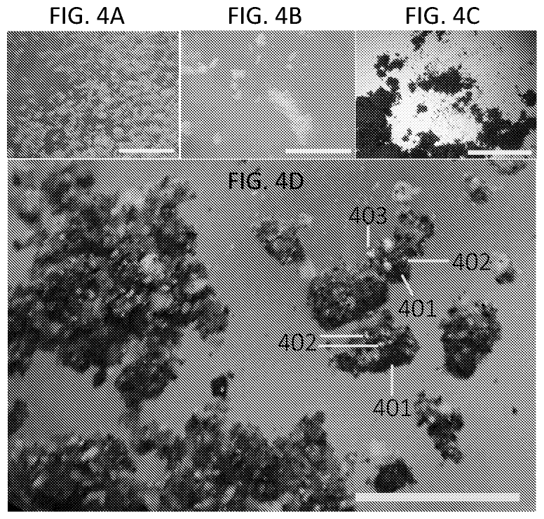

The formulation is prepared such that Erioglaucine is mixed with microcrystalline cellulose 102 (MCC) and MgCO₃. Erioglaucine bulk powder is mixed at 40% by weight, MCC at 30% by weight and MgCO₃ is at 30% by weight. Hence 1.5 g of MCC is placed in a glass bottle, followed by 2 g of Erioglaucine in the glass bottle and then 1.5 g of MgCO₃ in the glass bottle. The contents are mixed for 15 minutes using a Turbla mixer. FIG. 4 shows images of the component powders of the formulation and the final formulation after mixing. FIG. 4 shows clear mechanofusion of the powders with the MgCO₃ coating and adhering to the Erioglaucine particles. For the nasal formulation, this powder would be administered intranasally. For the oral formulation, this powder would be compressed into tablet form or put in a capsule. For the sublingual formulation, this powder would be compressed into a sublingual tablet.

### Example 2: Acidic Gas-Release Triggering Solution Spray (Activation Agent)

A strong mineral acid or a weak organic acid such as acetic acid may be used to prepare triggering solutions of different pH values. A pH 4.5 spray is prepared using commercial 1M hydrochloric acid and distilled water. First, the 1M HCl is diluted to 0.01M by adding 0.1 mL of 1M is diluted to 10 mL of total solution. From that 31.6 μL of the 0.01M HCl solution is diluted to 10 mL of total solution to yield a 10 mL solution of pH 4.5. A few drops of solution is put on pH paper to confirm the pH. The solution is loaded into a spray bottle that releases a 140 μL volume per spray. For the nasal dosage form this would be for the administration of the activation agent triggering spray after the administration of the nasal powder. For the oral or sublingual dosage form, such as in tablet form, this would mimic exposure to gastric or salivary fluids.

### Example 3: Franz Cell Transport Measurement

The Franz cell diffusion apparatus is commonly used in the field to evaluate transport of pharmaceutical compositions in vitro and ex vivo. [Laurent Salade*, Nathalie Wauthoz, Jonathan Goole, Karim Amighi, International Journal of Pharmaceutics 561 (2019) 47-65.] A vertical Franz diffusion cell is used (0.64 cm² area) is obtained from PermeGear, Inc. (Hellertown, PA, USA) FIG. 56. It is used to evaluate molecular transport across a validated synthetic permeation barrier, with the validated synthetic barrier used to model biological barriers (Polysulfone polymer membranes, PermeGear, Inc.). The synthetic barrier membranes are inserted between the Franz cell's donor and receptor compartments. The receptor compartment is filled with water and maintained under stirring. 30 mg of the powder formulation is then placed in the donor compartment within a Franz cell.

For the reference set up no activation agent solution is added. Video capture of the receptor chamber is done with color analysis conducted on the receiving solution at set time points to determine rate and level of transport across the model biological barrier. For the experimental setup, after 30 mg of the powder formulation is placed within the donor chamber a single 140 μL volume of activation agent solution is sprayed on the powder formulation in the donor chamber. The solution is sprayed immediately after placing the powder formulation in the donor chamber.

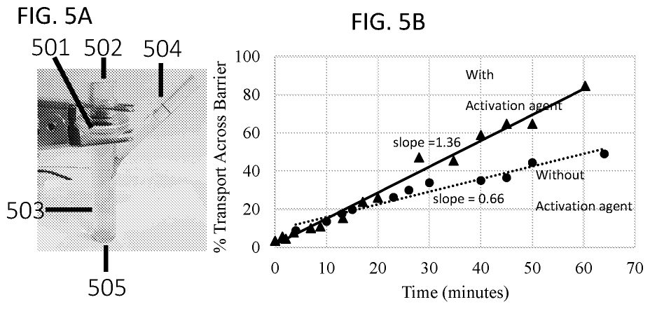

In FIG. 5, the data for the permeation of the Erioglaucine containing composition across a model of a biological barrier when an activation agent is applied to the propulsion agent containing composition with propulsion, and when no activation agent is applied to the same composition without propulsion is shown. With the addition of the activation agent, the composition is detected in the receptor chamber immediately (within the first second) after application of the activation agent, where the activation agent is sprayed onto the composition immediately after placing it in the donor chamber. In cases where no activation agent is applied, and hence without propulsion, transport across the membrane only starts to occur after 4 minutes of adding the composition to the donor chamber. The overall transport rate across the membrane is indicated by the slopes of the plots of the percentage Erioglaucine permeated as a function of time. The rate of transport of the composition with the application of the activation agent is more than 2 times faster than without the application of the activation agent.

### Example 4: Formulation Using a Soluble Propulsion Agent

The formulation is prepared such that Erioglaucine is mixed with microcrystalline cellulose 102 (MCC) and sodium bicarbonate NaHCO₃. Erioglaucine bulk powder is mixed at 40% by weight, MCC at 30% by weight and NaHCO₃ is at 30% by weight. Hence 1.5 g of MCC is placed in a glass bottle, followed by 2 g of Erioglaucine in the glass bottle and then 1.5 g of NaHCO₃ in the glass bottle. The contents are mixed for 15 minutes using a Turbla mixer.

### Example 5: Powder Formulation Incorporating a Powder Acid

In this embodiment the powder formulation contains a powder acid. Powder acids can be incorporated in formulations containing both soluble and insoluble propulsion agents. The formulation is prepared such that Erioglaucine is mixed with microcrystalline cellulose 102 (MCC), citric acid powder and MgCO₃. Erioglaucine bulk powder is mixed at 40% by weight, MCC at 25% by weight, citric acid powder at 5% by weight and MgCO₃ is at 30% by weight. Hence 1.5 g of MCC is placed in a glass bottle, followed by 2 g of Erioglaucine in the glass bottle, followed by 0.25 g of citric acid powder and then 1.5 g of MgCO₃ in the glass bottle. The contents are mixed for 15 minutes using a Turbla mixer.

### Example 6: Water as the Activation Agent

Water can serve as the activation agent in the case of a soluble propulsion agent in the presence or absence of a powder acid in the formulation. Water can serve as an activation agent when a propulsion agent is insoluble in water when a powder acid is present in the formulation. The presence of a powder acid in the formulation means that using water alone as the triggering spray would yield a low pH within the nasal cavity and allow the release of gas and the propulsion and embedding of the active ingredient. As such, 140 μL volume of water as an activation agent is sprayed intranasally onto the powder formulation after administration into the nasal cavity. Water is naturally present in the GI tract for activation of an oral formulation or in the mouth for a sublingual tablet.

### Example 7: BCS Class I: Warfarin

In this embodiment the use of a BCS Class I drug is described.

### Powder Formulation: Granules Incorporating Gas Producing Agent.

Warfarin (WF) bulk powder is mixed with MCC and MgCO₃. WF at 50% by weight, MCC at 25% by weight and MgCO₃ at 25% by weight. Hence 200 mg of MCC, followed by 400 mg of WF and then 200 mg of MgCO₃ would be placed in a 2 oz glass bottle and blended in a Turbla mixer for 15 minutes. The approximate particle sizes of the drug (WF), MCC and MgCO₃ are in a range of 5-10 μm, 50 μm and 2-5 μm respectively. Hence upon mixing, the WF and magnesium carbonate mechanofuse to the surface of the MCC. To ensure full adhesion the mix would be then manually granulated using a mortar and pestle. Hence granules that incorporate the gas producing agent MgCO₃ are produced. The mix would then be sieved on a 100 mesh (150 μm).

### Example 8: Powder Formulation: Granules Only Coated by Gas Releasing Agent

In addition to the direct mix of actives and excipients, including the gas releasing agent, another structure of the powder used for the nasal formulation includes coating granules, aggregates or microparticles containing the API with the gas-producing agent. The granules or aggregates themselves may either contain gas producing agent or may only be coated by it. Granules or aggregates may be composed of neat API or microspheres of API obtained using any of the methods known by those skilled in the art, such as spray drying lyophilization, among other where the microspheres may either contain excipients or no excipients.

### Example 9: Franz Cell Diffusion

A vertical Franz diffusion cell (0.64 cm² area) with sheep nasal mucosa as the barrier to evaluate permeation of the various formulations. A spray of pH 4.5 is prepared using hydrochloric acid and distilled water as described in Example 2. For the reference or control experiment, no activation agent solution is added.

At set time points, 0.4 ml of receptor solution is withdrawn, and the receptor compartment refilled with an equivalent volume of fresh PBS. At the end of the experiment, the residual formulation on the membrane is quantitatively recovered by rinsing the donor compartment with PBS. All samples would be analyzed by HPLC as described in Example 11.

### Example 10: Animal Studies

The evaluation of bioavailability of the drug in the presence and absence of propulsive gas-production can be done in animal studies. Three to four male Wister rats weighting 250 g each are used for each of the formulation with gas production and that in the absence of gas production for comparison. The rats are anesthetized with intraperitoneal injections of pentobarbital sodium (52 mg/kg), and the right femoral artery is cannulated with polyethylene tubing. The Dry Powder Insufflator™ Model DP-4 from Penn-Century (Penn-Century Inc., Philadelphia, PA), developed for different animal models is used to deliver 1 mg of powder into the nasal cavity of rats. The activation agent solution is administered to the rats using a micropipette into the nasal cavity. The animals are then allowed to reach consciousness and blood samples are collected at set times after drug administration. Blood samples are centrifuged at 5,000 g for 5 min to obtain the plasma. All samples would be analyzed by HPLC as described in Example 11.

### Example 11: HPLC Analysis

HPLC analysis conducted for Franz cell diffusion samples and rat in vivo samples could use an HPLC system (LC-20, Shimadzu, Kyoto, Japan). An ODS column (Wakopak, 5 mm, 4.6×150 mm, Wako Pure Chemical Industry, Osaka, Japan) is used with a mobile phase of 10 mM tetrabutyl ammonium in 10 mM phosphate buffer (pH 7.4) with methanol at a 1:1 ratio. The flow rate is 0.5 mL/min and the detection utilized an excitation at 310 nm and an emission at 390 nm. For blood plasma samples, acetonitrile (1,000 μL) is added to the plasma (100 μL) for deproteinization. The mixture is vortexed for 10 min and centrifuged at 5,000 g for 5 min. The supernatant (1,000 μL) is evaporated and dried at 60° C. The residue would be reconstituted in 100 μL of the mobile phase for use in analysis.

### Example 12: BCS Class II: Piroxicam

In this embodiment the use of a BCS Class I drug is described.

### Powder Formulation

Piroxicam (PXC) bulk powder is mixed with MCC 102 and MgCO₃. PXC is at 50% by weight, MCC at 25% by weight and MgCO₃ is at 25% by weight. Hence 200 mg of MCC, followed by 400 mg of PXC and then 200 mg of MgCO₃ would be placed in a 2 oz glass bottle and blended in a Turbla mixer for 15 minutes.

### Example 13: Animal Studies

The evaluation of bioavailability of the drug in the presence and absence of propulsive gas-production can be done in animal studies. Three to four male Wister rats weighting 250 g each are used for each of: the formulation with gas production, and that in the absence of gas production for comparison. The rats are anesthetized with intraperitoneal injections of pentobarbital sodium (52 mg/kg), and the right femoral artery is cannulated with polyethylene tubing. The Dry Powder Insufflator™ Model DP-4 from Penn-Century (Penn-Century Inc., Philadelphia, PA), developed for different animal models is used to deliver 1 mg of powder of the formulation from Example 13 into the nasal cavity of rats. The gas-release triggering solution (activation agent) is administered to the rats using a micropipette into the nasal cavity. The animals are then allowed to reach consciousness and blood samples are collected at set times after drug administration. Blood samples are centrifuged at 5,000 g for 5 min to obtain the plasma.

### Example 14: HPLC Analysis

HPLC analysis can be conducted using an HPLC system (LC-20, Shimadzu, Kyoto, Japan). An ODS column (Wakopak, 5 mm, 4.6×150 mm, Wako Pure Chemical Industry, Osaka, Japan) with a mobile phase composed of 50 mM KH2PO4 buffer (pH 2.5) and acetonitrile in a ratio of 68:32 would be needed. The flow rate set at 1.0 mL/min and photometric detection done at 326 nm. For blood plasma samples, acetonitrile (1,000 μL) is added to the plasma (100 μL) for deproteinization. The mixture is vortexed for 10 min and centrifuged at 5,000 g for 5 min. The supernatant (1,000 μL) is evaporated and dried at 60° C. The residue reconstituted in 100 μL of the mobile phase for use in analysis.

### Example 15: BCS Class III: Zolmitriptan. Zolmitriptan-Chitosan Mucoadhesive Microparticles Produced Using Spray Drying (Mg to Gram Quantities)

In this embodiment the use of a BCS Class III drug is described.

The drug zolmitriptan is used for the treatment of migraines. The typical therapeutic dose for adults and children 12 years and older is 2.5 to 5 mg, with no more than 10 mg in a 24-hour period.

Zolmitriptan may be formulated as a neat powder or within a mucoadhesive microparticle to enhance bioavailability. The latter is described herein. Formulations may contain one or more diluents such as mannitol or lactose or none.

Microparticles can be prepared using spray drying. A wide range of ratios of zolmitriptan (90-10%) to chitosan glutamate (10-90%) may be used to prepare microparticles. In this example a 1:1 ratio containing 50% zolmitriptan to 50% chitosan glutamate mucoadhesive agent is described.

375 mg of the drug (Haorui Pharma-Chem Inc. New Jersey, USA) and 375 mg of the chitosan glutamate Protasan UP G 213 (CG213; Mw, 200-600 kDa; deacetylation degree, 75-90%; from NovaMatrix/FMC Biopolymer in Sandvika, Norway) are dissolved in 250 ml of distilled water. The pH is adjusted to 5.0 using 0.25% (v/v) acetic acid to assist the dissolution of the components. A Büchi Mini Spray Dryer B-290 (Büchi Labortechnik AG, Switzerland) an open-cycle system with a pressure nozzle (co-current flow) is used with air as the drying medium. The conditions of operation are an inlet temperature of 160° C., an outlet temperature of 82-85° C., an aspiration rate of 100%, air flow of 357 l/h and a solution feed rate of 5 ml/min.

### Example 16: Large Scale Production of Zolmitriptan-Chitosan Mucoadhesive Microparticles

375 g of the drug Zolmitriptan (Haorui Pharma-Chem Inc. New Jersey, USA) and 375 g of the chitosan glutamate Protasan UP G 213 (CG213; Mw, 200-600 kDa; deacetylation degree, 75-90%; from NovaMatrix/FMC Biopolymer in Sandvika, Norway) are dissolved in 250 L of distilled water. The pH is adjusted to 5.0 using 0.25% (v/v) acetic acid to assist the dissolution of the components. A Büchi Mini Spray Dryer B-290 (Büchi Labortechnik AG, Switzerland) an open-cycle system with a pressure nozzle (co-current flow) is used with air as the drying medium. The conditions of operation are an inlet temperature of 160° C., an outlet temperature of 82-85° C., an aspiration rate of 100%, air flow of 357 l/h and a solution feed rate of 5 ml/min.

### Example 17: Blending and Agglomeration of Zolmitriptan-Chitosan Microparticles Incorporating the Propulsion Agent in the Agglomerate

Wet or dry agglomeration may be used as is known by those skilled in the art. It is possible to agglomerate the particles in the spray drying step by recycling fine particles separated from the exhaust air, adding them back into the dryer to generate agglomerates. In this embodiment, an agitation-based agglomeration approach is described.

700 mg of spray dried powder, 700 mg of a diluent, in this case microcrystalline cellulose, and 700 mg of the gas producing agent magnesium carbonate MgCO₃ are blended in a TurbulaVR blender (WAB, Muttenz, Switzerland) for 10 minutes. It should be noted that many ratios of the three components may be used such that the amount of active ingredient containing microparticles may range from (10-90%), the amount of diluent may range from 10-90%) And the amount of gas producing salt may range from (5-60%) For the three to come out to 100%. it should also be noted that any identity of diluent or combination of more than one diluent as well as any identity of gas producing salt mix of different gas producing agents may be used. The portion of the mixture partially adhered to the container's wall, is then gently scraped off with a spatula and the powder further mixed for 10 min. The mix is then placed on top of a 2-sieve nest (10 cm diameter sieves, Endecotts Ltd, London, UK; nominal aperture 0.300 and 0.150 mm, respectively) on a laboratory sieve shaker (Vibratory Sieve Shaker, Retsch VR, Haan, Germany). The system is vibrated for 5 min at an amplitude of ⅘. Reprocessing of non-agglomerated powder is repeated five times. The agglomerates retained on the 150 μm sieve are collected, weighed to calculate the percentage agglomeration yield (ratio between the weight of the agglomerates on the sieve and the amounts of materials processed), and stored in tightly closed containers to be used for further studies.

### Example 18: Dry Granulation Using Low Force Roller Compaction of Zolmitriptan-Chitosan Mucoadhesive Microparticles Incorporating the Propulsion Agent in the Granule

Utilizing the microparticles produced in Example 16, and prior to roller compaction a pre-granulation formulation of the components is first blended. 500 grams of the zolmitriptan-chitosan mucoadhesive microparticles, 250 grams of microcrystalline cellulose, 250 grams of magnesium carbonate MgCO₃ and 25 grams of magnesium stearate are blended for 20 minutes in a Bohle blender. The powder blend is then fed into a Gerteis mini-pactor roller compactor with a roll speed of 6 rpm, roll force of 2 tons using a rotating impeller milling method and a granulator screen of mesh 12 (U.S. standard). While roller compaction is not often used for nasal formulations, with emphasis placed on rapid disintegration, the presence of the gas producing salt within the granules will assist in rapid disintegration and penetration into the nasal lining. For the nasal formulation, this powder would be administered intranasally followed by an acidic spray as that described in Example 2. For the oral formulation, this powder would be compressed into tablet form (to be swallowed or sublingual) or put in a capsule for administration.

### Example 19: Granules of Zolmitriptan-Chitosan Microparticles Both Containing and Coated by Gas Releasing Propulsion Agent

Granules obtained from Example 18 are then blended with MgCO₃. 250 grams of Zolmitriptan-Chitosan Microparticle Granules containing the propulsion agent.

### Example 20: Insulin

The delivery of macromolecules and biologics that have been developed remains largely parenteral and hence alternate non-invasive forms of delivery are desired. Insulin here serves as an example of a macromolecular biologic. It is critical for individuals with diabetes.

### Powder Formulation

Insulin from porcine pancreas (Sigma chemical company) is blended with microcrystalline cellulose 102 (MCC) and MgCO₃. Insulin is at 20% by weight, MCC at 70% by weight and MgCO₃ is at 10% by weight. Hence 7 g of MCC, followed by 2 g of Insulin and then 1 g of MgCO₃ would be placed in a 4 oz glass bottle and blended in a Turbla mixer for 15 minutes. The resultant blend is loaded into and insufflator for administration to rats.

### Example 21: Animal Studies_Insulin

The evaluation of bioavailability of the drug in the presence and absence of propulsive gas-production can be done in animal studies. Three to four male Wister rats weighting 250 g each are used for each of: the formulation with gas production, and that in the absence of gas production for comparison. The rats are fasted for 10 hours and anesthetized with intraperitoneal injections of pentobarbital sodium (52 mg/kg), and the right femoral artery is cannulated with polyethylene tubing. The Dry Powder Insufflator™ Model DP-4 from Penn-Century (Penn-Century Inc., Philadelphia, PA), developed for different animal models is used to deliver 1 mg of powder of the formulation from Example 20 into the nasal cavity of rats. The activation agent solution is administered to the rats using a micropipette into the nasal cavity. The animals are then allowed to reach consciousness and blood samples are collected at set times after drug administration. Blood samples are centrifuged at 5,000 g for 5 min to obtain the plasma. Plasma samples are stored frozen at −40° C. until needed for the assay. Glucose level analysis to determine Changes in plasma glucose levels as a function of time are determined by using a Wake Glucose B-Test Kit (Wako Pure Chemical Industries, Ltd.) according to the glucose oxidase method.

What is claimed is:

1. A pharmaceutical composition comprising a pharmaceutical agent admixed with or coated with a propulsion agent comprising a component capable of evolving a propulsion gas upon contact with an activation agent; thereby allowing the pharmaceutical agent to embed within or penetrate a biological tissue or biological barrier.

2. The pharmaceutical composition of claim 1, wherein the propulsion gas evolved is a biologically benign gas or a gas, that at the evolved amount, does not pose a hazard to the health of the patient, be it a human or an animal.

3. The pharmaceutical composition of claim 1 or 2, wherein the propulsion gas is one or more of CO₂, N₂, O₂, NO₂, H₂, H₂O vapor and SO₂, and any combination thereof.

4. The pharmaceutical composition of any one of claims 1-3, wherein the propulsion gas is carbon dioxide.

5. The pharmaceutical composition of claim 1, wherein the component of the propulsion agent capable of evolving a propulsion gas comprises a carbonate or bicarbonate compound.

6. The pharmaceutical composition of any one of claims 1-3, wherein the propulsion gas is a nitrogen or nitrogen dioxide.

7. The pharmaceutical composition of any one of claims 1-3 and 6, wherein the propulsion agent capable of evolving a propulsion gas comprises nitrate or nitrite component.

8. The pharmaceutical composition of any one of claims 1-3, wherein the propulsion gas is sulfur dioxide.

9. The pharmaceutical composition of any one of claims 1-3 and 8, wherein the propulsion agent capable of evolving a propulsion gas comprises a sulfite compound.

10. The pharmaceutical composition of any one of claims 1-3, wherein the propulsion gas is oxygen, hydrogen or water vapor.

11. The pharmaceutical composition of any one of claim 1 or 2, wherein the activation agent comprises water.

12. The pharmaceutical composition of any one of claim 1 or 2, wherein the activation agent is an acidic solution comprising a pH at or below about 7, optimally in the range of about pH 2-6.5.

13. The pharmaceutical composition of any one of claim 1 or 2, wherein the activation agent is a solution of pH that ranges from about 1 to about 14.

14. The pharmaceutical composition of claim 1, wherein the gas-producing propulsion agent exists as or in one of the following structures or a combination thereof:
Admixed with or coating the active pharmaceutical ingredient
Within the particles containing the active pharmaceutical ingredient as a result of co-processing, such as co-spray drying with the active pharmaceutical ingredient and any other excipient
Incorporated within granules or aggregates of neat active pharmaceutical ingredient particles or active pharmaceutical ingredient-containing particles along with any other excipient Incorporated intergranular or coating the granules or aggregates of neat active pharmaceutical ingredient particles or active pharmaceutical ingredient-containing particles

15. A method for treating a disease or disorder in a subject in need thereof comprising administering a therapeutically effective amount of the pharmaceutical composition of any one of claims 1-14 to a subject in need thereof.

16. The method of claim 15, wherein the pharmaceutical composition is administered nasally.

17. The method of claim 15, wherein the pharmaceutical composition is administered orally.

18. The method of claim 15, wherein the pharmaceutical composition is administered sublingually.

19. The method of claim 15, wherein the pharmaceutical composition is administered via a route that is rectal, transdermal, vaginal, ocular, or the like.

20. The method of any one of claims 15-19, further comprising administering an activation agent that contacts the pharmaceutical composition, thereby promoting evolution of the propulsion gas from the pharmaceutical composition.

21. The method of claim 20, wherein evolution of the propulsion gas promotes embedding or penetration of all or a part of the pharmaceutical composition in a biological tissue or a biological barrier.

22. The method of claim 21 wherein embedding or penetration occurs within the nasal tissue.

23. The method of claim 22 wherein the embedding or penetration promotes nose-to-brain delivery of the pharmaceutical agent.

24. The method of claim 22, wherein the embedding or penetration promotes systemic delivery of the pharmaceutical agent.

25. The method of claim 22, wherein the embedding or penetration promotes delivery to non-brain target organs.

26. The method of claim 21 wherein the embedding or penetration occurs within the gastrointestinal lining or the sublingual lining.

27. The method of any one of claims 15, 21, and 22, wherein the pharmaceutical composition of claim 1 of a structure of claim 23; is administered intranasally followed, in sequence, by the administration of the activation agent (e.g. water or an acidic solution), to the same location within the nasal cavity to allow for the contact of the propulsion agent and the activation agent and the release of the propulsion gas.

28. The method of claim 27, wherein evolution of the propulsion gas promotes embedding or penetration of all or a part of the pharmaceutical composition in nasal tissue.

29. The pharmaceutical compositions of claim 27, wherein the pharmaceutical composition nasal dosage form can be in the form of powders, sprays, emulsions, suspensions, and the like, which contain the active ingredient(s) in a mixture with non-toxic pharmaceutically acceptable excipients.

30. The pharmaceutical composition of any one of claims 1-14 wherein the pharmaceutical composition is an internasal powder comprising:
One or more active pharmaceutical agents in an amount of up to about 99.9% by weight with a common range of about 10% to 99% by weight.
One or more gas producing propulsion agent compounds (e.g. carbonates, bicarbonates or the like) such that their total weight percent ranges from about 0.1% to about 95% by weight, optimally in the range of 0.5% to 50% by weight.
One or more acids in powder form, in the range of about 0% to 85% by weight, optimally in the range of 0 to 50% by weight.
One or more mucoadhesive agents (e.g. hydroxypropyl cellulose, hydroxypropyl methyl cellulose, hydroxypropyl ethyl cellulose, carboxymethylcellulose, carboxymethyl cellulose, pectin, chitosan, carboxymethyl chitosan, and the like) in the range of about 0 to about 80% by weight and where they may be admixed with the active ingredients; or co-formulated and co-processed with the active ingredients within particles, or both.
One or more diluents (e.g. microcrystalline cellulose, lactose, mannitol, starch and the like) at about 0% to about 95% by weight.
One or more disintegrants or super disintegrants (e.g. sodium starch glycolate, croscarmellose sodium, crospovidone and the like) to assist the breakup of any granules between about 0% and 25% by weight.
One or more lubricants (e.g. magnesium stearate, stearic acid, hydrogenated vegetable oil and the like) at between about 0% and 2% by weight is typically used only if a granulation step, such as roller compaction, is needed.
One or more absorption enhancers from about 0% to 50% by weight,
wherein the amounts by weight are based on the total weight of the composition and do not exceed 100% by weight

31. The pharmaceutical composition of any one of claims 1-14 wherein the pharmaceutical composition is an internasal powder comprising:
One or more active pharmaceutical agents in an amount with the range of about 50% to about 99% by weight.
One or more gas producing propulsion agent compounds (e.g. carbonates, bicarbonates or the like) such that their total weight percent ranges from about 1% to about 50% by weight.
wherein the amounts by weight are based on the total weight of the composition and do not exceed 100% by weight

32. The pharmaceutical composition of any one of claims 1-14 wherein the pharmaceutical composition is an internasal powder comprising:
One or more active pharmaceutical agents in an amount with the range of about 50% to about 99% by weight.
One or more gas producing propulsion agent compounds (e.g. carbonates, bicarbonates or the like) such that their total weight percent ranges from about 1% to about 50% by weight.
One or more diluents (e.g. microcrystalline cellulose, lactose, mannitol, starch and the like) at about 1% to about 50% by weight.
wherein the amounts by weight are based on the total weight of the composition and do not exceed 100% by weight

33. The pharmaceutical composition of any one of claims 1-14 wherein the pharmaceutical composition is an internasal powder comprising:
One or more active pharmaceutical agents in an amount with the range of about 50% to about 99% by weight.
One or more gas producing propulsion agent compounds (e.g. carbonates, bicarbonates or the like) such that their total weight percent ranges from about 1% to about 50% by weight.
One or more diluents (e.g. microcrystalline cellulose, lactose, mannitol, starch and the like) at about 0% to about 50% by weight.
One or more mucoadhesive agents in the range of about 1 to about 50% by weight and where they may be admixed with the active ingredients; or co-formulated and co-processed with the active ingredients within particles, or both.
wherein the amounts by weight are based on the total weight of the composition and do not exceed 100% by weight

34. The pharmaceutical composition of any one of claims 1-14 or 30-33 wherein the pharmaceutical composition is an internasal spray.

35. The method of claim of any one of claims 27-29, wherein the activation agent is a liquid nasal spray of any one of claims 11-13.

36. A method of claim 27-29 wherein the time duration between the administration of the pharmaceutical composition containing the gas producing propulsion agent nasally of any one of claims 1, 30-34 and the activation agent spray of any one of claims 11-13 is fractions of a second.

37. A method of claim 27 wherein the time duration between the administration of the pharmaceutical composition of internasal powder of any one of claims 1-13 or 30-34 and the activation agent spray of any one of claims 11-13 is a second or seconds.

38. A method of claim 27 wherein the time duration between the administration of the pharmaceutical composition of internasal powder of any one of claims 1-13 or 30-34 and the activation agent spray of any one of claims 11-13 is minutes.

39. A method of claim 26 wherein the time duration between the administration of the pharmaceutical composition of internasal powder of any one of claims 1-13 or 30-34 and the activation agent spray of any one of claims 11-13 is an hour or more than an hour.

40. A method of claim 27 wherein more than one administration of the activation agent is administered, wherein administrations following the initial administration of an activation agent occurs at a time ranging from fractions of a second to more than an hour.

41. A method of claim 27 wherein the administration of the pharmaceutical composition of any one of claims 1-13 or 30-34 and the activation agent spray of any one of claims 11-13 is done using a single device, where both the pharmaceutical composition and the activation agent are housed within the same device.

42. A method of claim 27 wherein the administration of the pharmaceutical composition of any one of claims 1-13 or 30-34 and the activation agent spray of any one of claims 11-13, is done using a kit, where the pharmaceutical composition and the activation agent are housed within separate devices.

43. The method of claims 15, 20 and 21, wherein the pharmaceutical composition of claim 1 is administered orally, wherein the activation agent is any one of claims 11-13, (e.g. water, an acidic solution or the like) is already present within the gastrointestinal tract and does not require separate administration.

44. The method of claim 43, wherein the pharmaceutical composition is formulated for oral administration, wherein the oral dosage forms may comprise tablets, capsules, a liquid solution or suspension, a powder, or liquid or solid crystals.

45. The pharmaceutical composition of any one of claims 1-14 wherein the pharmaceutical composition is a powder for oral or sublingual administration in tablet, capsule or powder form comprising:
One or more active pharmaceutical ingredients in an amount of up to about 95% by weight and more commonly 2 to 80% by weight
One or more diluents (e.g. microcrystalline cellulose, lactose, mannitol, starch and the like) at about 20% to about 90% by weight.
One or more disintegrants or super disintegrants (e.g. sodium starch glycolate, croscarmellose sodium, crospovidone and the like) to ensure the breakup of a tablet formulation at between about 0% and 25% by weight with most disintegrants within 2% and 15%.
One or more lubricants, (e.g. magnesium stearate, stearic acid, hydrogenated vegetable oil and the like) at between about 0% and 3% by weight with most between about 0.1% and 1.5%.
One or more anti-adherents and glidants at between about 0% and 3%.
One or more gas producing propulsion agents (e.g. carbonate, bicarbonate and the like) from about 0.5% to about 95% by weight, optimally in the range of about 0.5% to about 50% by weight.
One or more acids in powder form at about 0% to 50% be weight to lower the local pH surrounding the tablet increasing the rate of gas production in the case of both soluble or insoluble gas producing propulsion agents.
One or more degradation enzyme inhibitors at 0 to 15%.
One or more permeation enhancers at 0 to 15%. wherein the amounts by weight are based on the total weight of the composition and do not exceed 100% by weight

46. The pharmaceutical composition of any one of claims 1-14 wherein the pharmaceutical composition is a powder for oral or sublingual administration in tablet, capsule or powder form comprising:
One or more active pharmaceutical ingredients in an amount of about 20 to 95% by weight
One or more diluents (e.g. microcrystalline cellulose, lactose, mannitol, starch and the like) at about 20% to about 80% by weight.
One or more disintegrants or super disintegrants at between about 2% and 15%.
One or more lubricants, at between about 0.1% and 3% by weight
One or more gas producing propulsion agents from about 5% to about 55% by weight
wherein the amounts by weight are based on the total weight of the composition and do not exceed 100% by weight

47. The method of claim 43, wherein the activation agent is water within the gastrointestinal tract (e.g. such as gastric fluids within the small intestine or stomach).

48. The method of claim 43, wherein the activation agent is an acidic aqueous solution within the gastrointestinal tract (e.g. such as gastric fluids within the stomach or certain locations within the intestine).

49. The pharmaceutical composition of any one of claims 45-46 in oral tablet form.

50. The pharmaceutical composition of any one of claims 45-46 in oral tablet form with a coating such as an enteric coating.

51. The pharmaceutical composition of any one of claims 45-46 loaded into a capsule form.

52. The pharmaceutical composition of any one of claims 45-46 loaded into an enteric capsule form.

53. The method of claims 15, 20 and 21, wherein the pharmaceutical composition of claim 1 is administered sublingually, wherein the activation agent is moisture already present within the mouth and does not require separate administration.

54. The method of claim 53 for sublingual administration, wherein the dosage forms may comprise tablets, films or sprays.

55. The method of any one of claim 53 or 54 wherein the pharmaceutical composition is a sublingual tablet of a powder or spray of composition of any one of claims 30-34 or 45-46.

56. A method of manufacturing a pharmaceutical composition of any one of claims 1, 30-33 or 45-46 comprising admixing a pharmaceutical agent with a propulsion coating configured to evolve a propulsion gas following contact with an activation agent.

57. A process for the manufacture of a pharmaceutical composition of any one of claims 1, 30-33 or 45-46 comprising:
i) Sieving each individual component to ensure the removal of any clumping
ii) Blending of the components which includes the active pharmaceutical ingredient(s) and the gas producing propulsion agent, and additional excipients.
wherein blending of the active pharmaceutical ingredient(s), any excipients and the gas producing propulsion agent leads to the mechanofusing of the different particles where adherence is due to surface forces which are especially magnified for particles on the micron, sub-millimeter and millimeter scale.

58. A process for the manufacture of a pharmaceutical composition of any one of claims 1, 30-33 or 45-46, with which to produce active pharmaceutical ingredient containing granules that both contain and are coated with the gas releasing propulsion agent comprising:
i) Sieving each individual component to ensure the removal of any clumping
ii) Blending of the pre-granulation components which includes the active pharmaceutical ingredient(s) and gas producing propulsion agent, and can include diluents, disintegrants and lubricants if needed.
iii) Dry Granulation, Wet Granulation or Agglomeration (e.g. roller compaction, fluid bed granulation, mechanical agglomeration using a sieve shaker or the like)
iv) Coating granules with gas producing propulsion agent. This may be done using simple blending via a mechanofusing mechanism or via other methods such as film coating. Optional additional excipients may be added here.
v) Sieving to remove any excess gas producing propulsion agent (with this being an optional step)
vi) Blending with any other excipients (optional step and may be done to assist in compaction into tablets for oral or sublingual administration)

59. A process for the manufacture of a pharmaceutical composition of any one of claims 1, 30-33 or 45-46, to produce active pharmaceutical ingredient containing granule that is just coated with the gas releasing propulsion agent comprising:
i) Sieving each individual component to ensure the removal of any clumping
ii) Blending of the pre-granulation components which includes the active pharmaceutical ingredient(s) and can include diluents, disintegrants and lubricants if needed.
iii) Dry Granulation, Wet Granulation or Agglomeration (e.g. roller compaction, fluid bed granulation, mechanical agglomeration using a sieve shaker or the like)
iv) Coating granules with gas producing propulsion agent. This may be done using simple blending via a mechanofusing mechanism or via other methods such as film coating. Optional additional excipients may be added here.
v) Sieving to remove any excess gas producing propulsion agent (with this being an optional step)
vi) Blending with any other excipients (optional step and may be done to assist in compaction into tablets for oral or sublingual administration)

60. A process for the production of oral or sublingual tablets of the pharmaceutical compositions of any one of claims 1, 30-33 or 45-46 by utilizing tablet compaction.

61. A process for the coating of tablets of the pharmaceutical compositions of any one of claims 1, 30-33 or 45-46. with an enteric coating or any other functional coating (e.g. such as by utilizing a film coating process).

62. A process for the production of enteric or non-enteric capsules of the pharmaceutical compositions of any one of claims 1, 30-33 or 45-46.

63. The pharmaceutical compositions of any one of claims 1, 30-34 or 45-46, wherein the at least one pharmaceutical active agent is a small molecule, a macromolecule, (e.g. a peptide, protein or biologic), a BCS I class drug, a BCS II class drug, a BCS III class drug or a BCS VI class drug.
